(19) United States
(12) Patent Application Publication
(10) Pub. No.: US 2025/0275742 A1
(43) Pub. Date: Sep. 4, 2025

(54) CALIBRATION DATA GENERATION DEVICE, CALIBRATION DATA GENERATION METHOD, PROGRAM, AND MEDICAL IMAGE CAPTURING APPARATUS

(71) Applicant: FUJIFILM Corporation, Tokyo (JP)

(72) Inventor: Tsuyoshi SUZUKI, Tokyo (JP)

(73) Assignee: FUJIFILM Corporation, Tokyo (JP)

(21) Appl. No.: 19/067,596

(22) Filed: Feb. 28, 2025

(30) Foreign Application Priority Data
Mar. 1, 2024 (JP) 2024-031694

(51) Int. Cl. A61B 6/58; A61B 6/04

(52) U.S. Cl. CPC A61B 6/582; A61B 6/0407

(57) ABSTRACT

There are provided a calibration data generation device, a calibration data generation method, a program, and a medical image capturing apparatus with which correction of a control parameter of an examination table according to a position where a sensor is installed is achieved. A calibration data generation device acquires first distance information from a sensor attached to a measurement room to a tabletop at a first up-down position, acquires first plane information at the first up-down position, acquires second distance information from the sensor to the tabletop at a second up-down position, acquires second plane information at the second up-down position, and generates calibration data for correcting a movement parameter of the tabletop by using the first distance information, the first plane information, the second distance information, and the second plane information.

### CROSS-REFERENCE TO RELATED APPLICATION

The present application claims priority under 35 U.S.C § 119(a) to Japanese Patent Application No. 2024-031694 filed on Mar. 1, 2024, which is hereby expressly incorporated by reference, in its entirety, into the present application.

### BACKGROUND OF THE INVENTION

### 1. Field of the Invention

The present disclosure relates to a calibration data generation device, a calibration data generation method, a program, and a medical image capturing apparatus.

### 2. Description of the Related Art

In a case in which a medical image of an examinee is captured using a medical image capturing apparatus such as an MRI apparatus, the examinee is imaged using a camera disposed on a ceiling or the like of an examination room, a posture of the examinee is detected based on a captured image, and a position, an orientation, and the like of the examinee are adjusted based on a detection result. Note that MRI is an abbreviation for Magnetic Resonance Imaging.

JP2019-088734A discloses a camera disposed on a ceiling of an operating room as a surgical device present in the operating room. JP2023-089745A discloses an MRI apparatus comprising an examination table camera that images an examinee placed on a tabletop. The apparatus disclosed in JP2023-089745A recognizes an examination site from image data obtained by using the examination table camera, and acquires a transport amount of the tabletop for the recognized examination site to reach a center of a magnetic field.

### SUMMARY OF THE INVENTION

An installation position of a sensor such as a camera used for detecting the examinee is designated in advance. However, equipment such as a fluorescent lamp, a fire alarm, and a frame of a hanging ejector may interfere with attachment of the sensor to the designated position. JP2019-088734A and JP2023-089745A do not include any description regarding a position where a camera is installed.

The present disclosure has been made in view of such circumstances, and an object of the present disclosure is to provide a calibration data generation device, a calibration data generation method, a program, and a medical image capturing apparatus with which correction of a control parameter of an examination table according to a position where a sensor is installed is achieved.

A first aspect of the present disclosure provides a calibration data generation device comprising: one or more processors; and one or more memories that store a program including one or more instructions executed by the one or more processors, in which the one or more processors execute the program stored in the one or more memories to acquire, by using a sensor attached to a measurement room in which an examination table having a tabletop on which an examinee is placed is installed, first distance information representing a distance from the sensor to the tabletop at a first up-down position in an up-down direction orthogonal to an installation surface on which the examination table is installed, acquire first plane information including information on a distance in a first plane orthogonal to the up-down direction at the first up-down position by using the sensor, acquire second distance information representing a distance from the sensor to the tabletop at a second up-down position different from the first up-down position in the up-down direction by using the sensor, acquire second plane information including information on a distance in a second plane orthogonal to the up-down direction at the second up-down position by using the sensor, and generate calibration data applied to correction of a movement parameter of the tabletop in the up-down direction and a movement parameter of the tabletop in a plane orthogonal to the up-down direction at any position in the up-down direction, by using the first distance information, the first plane information, the second distance information, and the second plane information.

With the calibration data generation device according to the first aspect, calibration data for correcting the movement parameter of the tabletop in the up-down direction orthogonal to the installation surface of the examination table and the movement parameter of the tabletop in the plane orthogonal to the up-down direction is generated based on the first distance information, the first plane information, the second distance information, and the second plane information acquired by using the sensor installed in the measurement room. As a result, the correction of the movement parameter of the tabletop according to the position where the sensor is installed is implemented.

The sensor may comprise a first sensor that measures the distance from the sensor to the tabletop, and a second sensor including an imaging device that generates a captured image of the plane orthogonal to the up-down direction.

In the plane orthogonal to the up-down direction, a front-rear direction in which the tabletop moves forward or backward and a lateral direction along a width of the tabletop orthogonal to the front-rear direction may be specified.

The term “acquire” is not limited to an aspect in which target information is acquired, and may include an aspect in which information serving as a basis for generating the target information is acquired and the target information is generated from the basis information.

A second aspect provides the calibration data generation device according to the first aspect, in which the first up-down position may correspond to a home position where the tabletop is at its lowest, and the second up-down position may correspond to a highest position where the tabletop is at its highest.

According to such an aspect, the first distance information, the first plane information, the second distance information, and the second plane information are acquired for the home position and the highest position where an error of the movement parameter according to the position where the sensor is installed is maximized. As a result, highly accurate calibration data can be generated.

A third aspect provides the calibration data generation device according to the first or second aspect, in which the one or more processors may acquire, as the calibration data, a combination of a slope and an intercept on a value axis of a linear function representing a relationship between a distance from the installation surface to the tabletop and a distance from the sensor to the tabletop.

According to such an aspect, calibration data suitable for an operation executed by the processor is generated.

The distance from the installation surface to the tabletop may be measured by an operator or may be automatically measured using a measuring device.

A fourth aspect provides the calibration data generation device according to any one of the first to third aspects, in which the one or more processors may acquire the first distance information and the second distance information measured using a distance measurement sensor that measures a distance to an object to be measured, and acquire a captured image of the first plane as the first plane information and acquire a captured image of the second plane as the second plane information, from an imaging device that images the plane orthogonal to the up-down direction.

In such an aspect, an imaging device may be applied as a distance-measuring sensor.

A fifth aspect provides the calibration data generation device according to the fourth aspect, in which the one or more processors may acquire, as the calibration data, a distance per pixel in the captured image relative to a distance from the installation surface to the tabletop.

According to such an aspect, the number of pixels in a captured image of the tabletop is converted into a distance on the tabletop.

A sixth aspect provides the calibration data generation device according to the fourth or fifth aspect, in which the one or more processors may acquire, as the calibration data, a distance between a center position of the captured image and a measurement center position of a measurement device that measures the examinee in a lateral direction orthogonal to a front-rear direction in which the tabletop moves forward or backward, for the second up-down position.

According to such an aspect, the correction of the movement parameter of the tabletop in the lateral direction orthogonal to the front-rear direction in which the tabletop moves forward or backward is implemented.

A seventh aspect provides the calibration data generation device according to the fourth or fifth aspect, in which the one or more processors may acquire, as the calibration data, a distance between a center position of the captured image and a measurement start position specified for the examinee in a front-rear direction in which the tabletop moves forward or backward, for the second up-down position.

According to such an aspect, the correction of the movement parameter of the tabletop in the front-rear direction in which the tabletop moves forward or backward is implemented.

An eighth aspect provides the calibration data generation device according to the fourth or fifth aspect, in which the one or more processors may acquire a captured image showing the entire tabletop as the captured image of the first plane.

In such an aspect, a captured image may be acquired in which a position of a headrest of the tabletop in the captured image is reflected at a specified position in the captured image.

A ninth aspect provides the calibration data generation device according to any one of the first to eighth aspects, in which the one or more processors may acquire, as the calibration data, a slope of a linear function representing a relationship between a position on the tabletop and a distance from the sensor to the tabletop.

According to such an aspect, in a case in which the sensor is installed in a state of being inclined with respect to the installation surface of the examination table, the correction of the movement parameter of the tabletop is implemented.

A tenth aspect of the present disclosure provides a calibration data generation method comprising: acquiring, by using a sensor attached to a measurement room in which an examination table having a placement surface on which an examinee is placed is installed, first distance information representing a distance from the sensor to a tabletop of the examination table on which the examinee is placed at a first up-down position in an up-down direction orthogonal to an installation surface on which the examination table is installed; acquiring first plane information including information on a distance in a first plane orthogonal to the up-down direction at the first up-down position by using the sensor; acquiring second distance information representing a distance from the sensor to the tabletop at a second up-down position different from the first up-down position in the up-down direction by using the sensor; acquiring second plane information including information on a distance in a second plane orthogonal to the up-down direction at the second up-down position by using the sensor; and generating calibration data applied to correction of a movement parameter of the tabletop in the up-down direction and a movement parameter of the tabletop in a plane orthogonal to the up-down direction at any position in the up-down direction, by using the first distance information, the first plane information, the second distance information, and the second plane information.

According to the calibration data generation method of the tenth aspect of the present disclosure, it is possible to obtain the same effects as those of the calibration data generation device according to the first aspect. The configuration requirements of the calibration data generation device according to the second to ninth aspects can be applied as configuration requirements of the calibration data generation method according to other aspects.

An eleventh aspect of the present disclosure provides a program causing a computer to implement: a function of acquiring, by using a sensor attached to a measurement room in which an examination table having a tabletop on which an examinee is placed is installed, first distance information representing a distance from the sensor to a tabletop of the examination table on which the examinee is placed at a first up-down position in an up-down direction orthogonal to an installation surface on which the examination table is installed; a function of acquiring first plane information including information on a distance in a first plane orthogonal to the up-down direction at the first up-down position by using the sensor; a function of acquiring second distance information representing a distance from the sensor to the tabletop at a second up-down position different from the first up-down position in the up-down direction by using the sensor; a function of acquiring second plane information including information on a distance in a second plane orthogonal to the up-down direction at the second up-down position by using the sensor; and a function of generating calibration data applied to correction of a movement parameter of the tabletop in the up-down direction and a movement parameter of the tabletop in a plane orthogonal to the up-down direction at any position in the up-down direction, by using the first distance information, the first plane information, the second distance information, and the second plane information.

With the program according to the eleventh aspect of the present disclosure, it is possible to obtain the same effects as those of the calibration data generation device according to the first aspect. The configuration requirements of the calibration data generation device according to the second to ninth aspects can be applied as configuration requirements of the program according to other aspects.

A twelfth aspect of the present disclosure provides a medical image capturing apparatus comprising: a measurement device that measures an examinee; a measurement data processing device that generates a medical image of the examinee based on a measurement result of the examinee; an examination table that is provided with a tabletop on which the examinee is placed; a sensor that is attached to a measurement room in which the examination table is installed and that detects the examinee; and a calibration data generation device that generates calibration data for correcting a movement parameter of the tabletop based on a position where the sensor is installed, in which the calibration data generation device includes one or more processors, and one or more memories that store a program including one or more instructions executed by the one or more processors, and the one or more processors execute the program stored in the one or more memories to acquire, by using the sensor attached to the measurement room in which the examination table on which the examinee is placed is installed, first distance information representing a distance from the sensor to the tabletop of the examination table on which the examinee is placed at a first up-down position in an up-down direction orthogonal to an installation surface on which the examination table is installed, acquire first plane information including information on a distance in a first plane orthogonal to the up-down direction at the first up-down position by using the sensor, acquire second distance information representing a distance from the sensor to the tabletop at a second up-down position different from the first up-down position in the up-down direction by using the sensor, acquire second plane information including information on a distance in a second plane orthogonal to the up-down direction at the second up-down position by using the sensor, and generate calibration data applied to correction of a movement parameter of the tabletop in the up-down direction and a movement parameter of the tabletop in a plane orthogonal to the up-down direction at any position in the up-down direction, by using the first distance information, the first plane information, the second distance information, and the second plane information.

With the medical image capturing apparatus according to the twelfth aspect of the present disclosure, it is possible to obtain the same effects as those of the calibration data generation device according to the first aspect. The configuration requirements of the calibration data generation device according to the second to ninth aspects can be applied as configuration requirements of the medical image capturing apparatus according to other aspects.

According to the present disclosure, calibration data for correcting the movement parameter of the tabletop in the up-down direction orthogonal to the installation surface of the examination table and the movement parameter of the tabletop in the plane orthogonal to the up-down direction is generated based on the first distance information, the first plane information, the second distance information, and the second plane information acquired by using the sensor installed in the measurement room. As a result, the correction of the movement parameter of the tabletop according to the position where the sensor is installed is implemented.

### BRIEF DESCRIPTION OF THE DRAWINGS

FIG. 1 is a perspective view schematically showing a configuration of an MRI apparatus.

FIG. 2 is a functional block diagram showing an electric configuration of the MRI apparatus shown in FIG. 1.

FIG. 3 is a block diagram showing a hardware configuration of an electric configuration of a console unit shown in FIG. 1.

FIG. 4 is a functional block diagram showing an electric configuration of an examination control device shown in FIG. 2.

FIG. 5 is a flowchart showing a procedure of an up-down movement calibration data generation method.

FIG. 6 is an explanatory diagram of an examination table height.

FIG. 7 is a schematic diagram of a screen on which a camera image is displayed.

FIG. 8 is a schematic diagram of another example of a screen on which a camera image is displayed.

FIG. 9 is a schematic diagram showing a specific example of a guide displayed in a superimposed manner on a camera image.

FIG. 10 is a graph showing an example of up-down movement calibration data.

FIG. 11 is an explanatory diagram of a mathematical equation representing a linear function of the graph shown in FIG. 10.

FIG. 12 is a flowchart showing a procedure of a front-rear lateral movement calibration data generation method.

FIG. 13 is an explanatory diagram of tabletop both-ends coordinate acquisition.

FIG. 14 is a graph showing an example of front-rear lateral movement calibration data.

FIG. 15 is an explanatory diagram of a mathematical equation representing a linear function of the graph shown in FIG. 14.

FIG. 16 is an explanatory diagram of an example of marking of a camera center at a home position.

FIG. 17 is an explanatory diagram of an example of marking of a camera center at a highest position of an examination table.

FIG. 18 is an explanatory diagram of acquisition of a deviation amount of tabletop center position.

FIG. 19 is an explanatory diagram of an example of marking after the tabletop is moved forward.

FIG. 20 is an explanatory diagram of an example of a difference value between a camera center and an ISO center.

FIG. 21 is a schematic diagram showing a specific example of calibration in a lateral direction and a front-rear direction.

FIG. 22 is a schematic diagram of a distance from a camera to the tabletop in a case in which the camera is inclined with respect to an installation surface.

FIG. 23 is an explanatory diagram showing an example of a measurement position on the tabletop.

FIG. 24 is a schematic diagram of calibration in a case in which the camera is inclined with respect to the installation surface.

### DESCRIPTION OF THE PREFERRED EMBODIMENTS

Hereinafter, a preferred embodiment of the present invention will be described in detail with reference to the accompanying drawings. In the following description and the accompanying drawings, the same constituent elements are denoted by the same reference numerals, and the duplicated description thereof is omitted. In addition, in the following embodiment, in a case in which a plurality of constituent elements are described and listed, it can be interpreted that at least one of the plurality of constituent elements is included.

### Configuration Example of MRI Apparatus

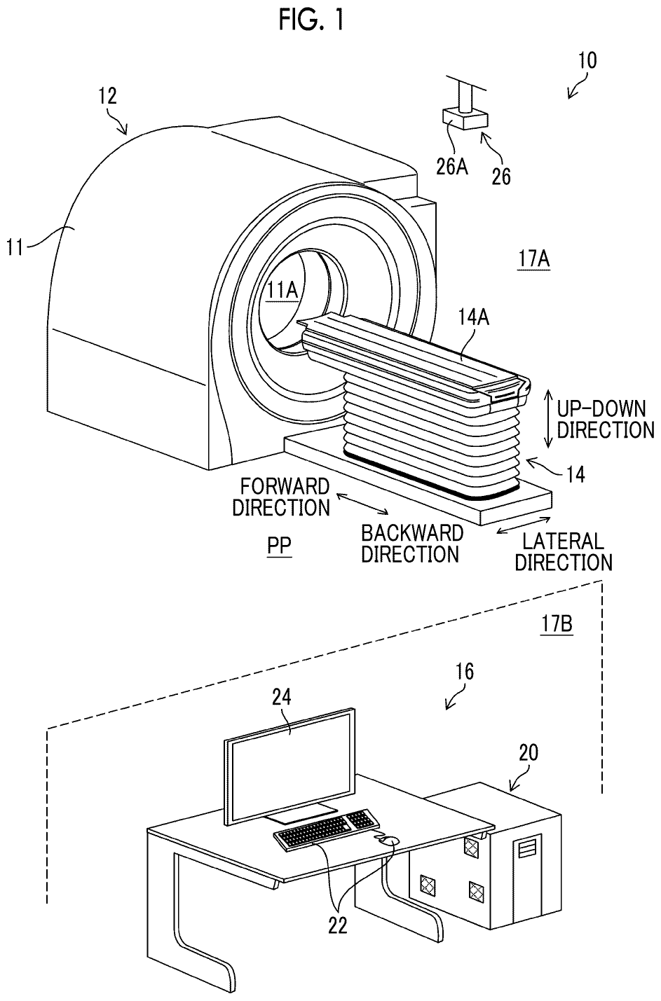

FIG. 1 is a perspective view schematically showing a configuration of an MRI apparatus. An MRI apparatus 10 comprises a measurement device 12 including a gantry 11, an examination table 14, and a console unit 16. The measurement device 12 and the examination table 14 are disposed in an imaging room 17A, and the console unit 16 is disposed in an operating room 17B. FIG. 1 shows a state in which the examination table 14 is set at a position where a medical image is captured for an examinee. The examination table 14 is configured to be movable manually or automatically on an installation surface PP.

The examination table 14 comprises a tabletop 14A on which the examinee undergoing an image diagnosis examination is placed. The tabletop 14A is movable in each of an up-down direction, a forward direction in which the tabletop 14A enters an imaging space 11A of the gantry 11, a backward direction in which the tabletop 14A exits the imaging space 11A, and a lateral direction orthogonal to the forward direction and orthogonal to the up-down direction, by using an examination table drive unit. Hereinafter, the forward direction and the backward direction may be referred to as a front-rear direction.

The console unit 16 comprises an examination control device 20, an input device 22, and a display device 24. The examination control device 20 functions as a control device that controls a control unit of a machine room to control each device of the measurement device 12 and the examination table 14 and that executes MRI imaging for acquiring an NMR signal. In addition, the examination control device 20 functions as a device that performs processing of various types of data including processing of reconstructing an image based on the NMR signal acquired from the measurement device 12 and communication processing through a network, display of a processing result, and storage of data.

The NMR signal obtained from the measurement device 12 is subjected to signal processing and then is subjected to digital image processing, and the reconstructed image is displayed on the display device 24 of the console unit 16. Note that NMR is an English abbreviation for Nuclear Magnetic Resonance.

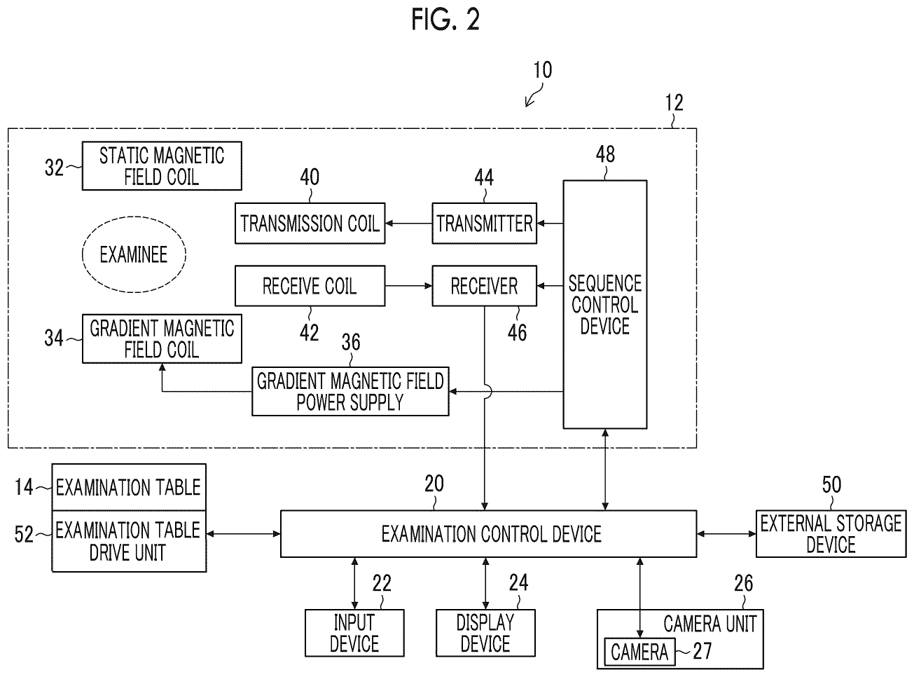

A camera unit 26 is installed on a ceiling of the imaging room 17A. The camera unit 26 comprises a camera and a case 26A in which the camera is accommodated. In FIG. 1, the camera is not shown. The camera is denoted by reference numeral 27 and is shown in FIG. 2.

The camera provided in the camera unit 26 images the examinee placed on the tabletop 14A and generates a camera image of the examinee. In addition, the camera provided in the camera unit 26 acquires distance information representing a distance from the camera to a subject such as the examinee.

The MRI apparatus 10 described in the embodiment is an example of a medical image capturing apparatus, and the examination control device 20 is an example of a measurement data processing device that generates a medical image of the examinee based on a measurement result of the examinee. The imaging room 17A described in the embodiment is an example of a measurement room. The camera unit 26 described in the embodiment is an example of a sensor attached to the measurement room.

### Electric Configuration of MRI Apparatus

FIG. 2 is a functional block diagram showing an electric configuration of the MRI apparatus shown in FIG. 1. The measurement device 12 comprises a static magnetic field coil 32, a gradient magnetic field coil 34, and a gradient magnetic field power supply 36. The static magnetic field coil 32 generates a static magnetic field in a space in which the examinee is placed. The gradient magnetic field coil 34 provides a magnetic field gradient with respect to the static magnetic field generated by the static magnetic field coil 32. The gradient magnetic field power supply 36 is a drive power supply of the gradient magnetic field coil 34.

The measurement device 12 comprises a transmission coil 40, a receive coil 42, a transmitter 44, and a receiver 46. The transmission coil 40 generates a high-frequency magnetic field in a measurement region of the examinee. The transmitter 44 supplies a pulse current, which is an excited current, to the transmission coil 40. The receive coil 42 receives the NMR signal generated from the examinee. The receiver 46 transmits the NMR signal received by using the receive coil 42 to the examination control device 20. The NMR signal may be referred to as an echo signal.

Any one of a vertical magnetic field method or a horizontal magnetic field method is applied to the MRI apparatus 10 depending on a direction of the static magnetic field to be generated. The static magnetic field coil 32 is adopted in various forms in accordance with the above-described magnetic field method. The gradient magnetic field coil 34 comprises a plurality of coils that generate gradient magnetic fields in three axial directions orthogonal to each other. The plurality of coils provided in the gradient magnetic field coil 34 are each driven by the gradient magnetic field power supply 36. Positional information is added to the NMR signal generated from the examinee due to the application of the gradient magnetic field.

Although FIG. 2 shows an aspect in which the transmission coil 40 and the receive coil 42 are individually provided, there may be an aspect in which one coil having a function of the transmission coil 40 and a function of the receive coil 42 is provided.

The measurement device 12 comprises a sequence control device 48. The sequence control device 48 controls the operations of the gradient magnetic field power supply 36 and the transmitter 44 to control generation timings of the gradient magnetic field and the high-frequency magnetic field. The sequence control device 48 controls the operation of the receiver 46 to control a reception timing of the NMR signal and to execute the measurement. A time chart of the control applied to the sequence control device 48 is referred to as an imaging sequence, is set in advance in accordance with the measurement, and is stored in a storage device or the like provided in the examination control device 20.

The examination control device 20 controls the operation of each unit of the measurement device 12 via the sequence control device 48. In addition, the examination control device 20 functions as an operation device that executes operation processing on the NMR signal received via the receiver 46 and the sequence control device 48 and that acquires an image signal of an imaging region determined in advance. The examination control device 20 may include the sequence control device 48. The examination control device 20 may be a device constituting the MRI apparatus 10 or may be an external device independent of the MRI apparatus 10.

The examination control device 20 is electrically connected to the input device 22, the display device 24, and an external storage device 50 in a communicable manner. The display device 24 displays a result of the operation processing executed by using the examination control device 20. For example, the display device 24 displays the reconstructed image reconstructed based on the NMR signal transmitted from the measurement device 12.

A liquid crystal display, an organic EL display, a projector, or the like can be applied as the display device 24. The display device 24 may be an appropriate combination of these. The organic EL may be referred to as OEL using an abbreviation for Organic Electro-Luminescence.

The input device 22 is an interface through which the operator inputs a condition applied to the measurement, a condition applied to the operation processing, parameters, and the like. Examples of the input device 22 include a keyboard and a mouse. The display device 24 and the input device 22 may be configured as an integrated configuration using a touch panel type display. The operator may use the input device 22 to input parameters such as the number of echoes to be measured, an echo time, and an echo interval.

The camera 27 provided in the camera unit 26 generates a camera image and distance information based on depth information of the camera 27, and transmits the camera image and the distance information to the examination control device 20. For example, the camera 27 may comprise a first camera that generates a camera image and a second camera that acquires distance information.

The first camera may comprise an image sensor that converts an optical image of the subject into an electric signal. Examples of the image sensor include a CCD image sensor and a CMOS image sensor. Note that CCD is an abbreviation for Charge Coupled Device, and CMOS is an abbreviation for Complementary Metal Oxide Semiconductor.

A stereo camera can be applied as the second camera. A passive method of imaging peripheral light can be applied to the stereo camera. An active method of emitting infrared light can be applied to the stereo camera. A ToF camera method can be applied to the second camera. Note that ToF is an abbreviation for Time-of-Flight. The first camera described in the embodiment is an example of an imaging device that generates a captured image, and the second camera is an example of a distance measurement sensor that measures a distance to an object to be measured.

The examination control device 20 detects a position of the examinee and a posture of the examinee using the camera image. The examination control device 20 applies the detection result and the distance information of the examinee to capturing the medical image of the examinee, such as the movement control of the tabletop 14A.

For example, in a case in which a height and a lateral position of the examinee are aligned with an ISO center specified as a center of the imaging space 11A of the gantry 11, the camera image and the distance information are used. The ISO center described in the embodiment is an example of a measurement center position of a measurement device that measures the examinee.

The external storage device 50 stores data used in various types of the operation processing executed by the examination control device 20, data derived as a result of the operation processing, the conditions applied to the operation processing, the parameters applied to the operation processing, a program for executing the operation processing, and the like. The external storage device 50 may implement a part of functions of an internal storage device of the examination control device 20, or the internal storage device of the examination control device 20 may implement a part of functions of the external storage device 50.

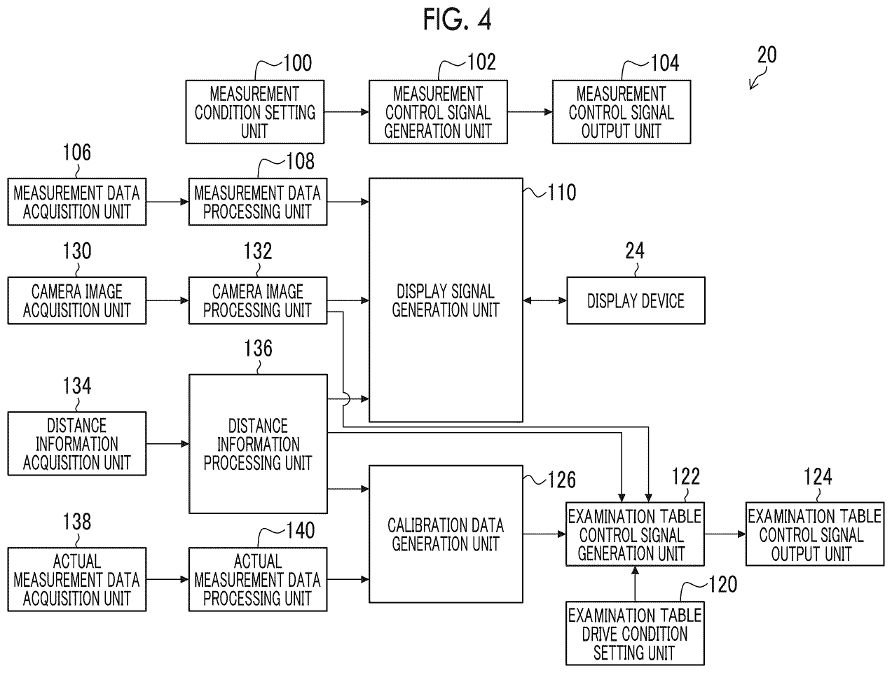

The MRI apparatus 10 comprises an examination table drive unit 52. The examination table drive unit 52 moves the tabletop 14A in each of the up-down direction, the front-rear direction, and the lateral direction. That is, the examination table drive unit 52 comprises a drive mechanism connected to the tabletop 14A and a drive source such as a motor connected to the drive mechanism. The examination table drive unit 52 is controlled by using an examination table drive control device. The drive mechanism and the drive source are not shown. In addition, the examination table drive control device is not shown in FIG. 2. Components of the examination table drive control device are shown in FIG. 4.

### Electric Configuration of Console Unit

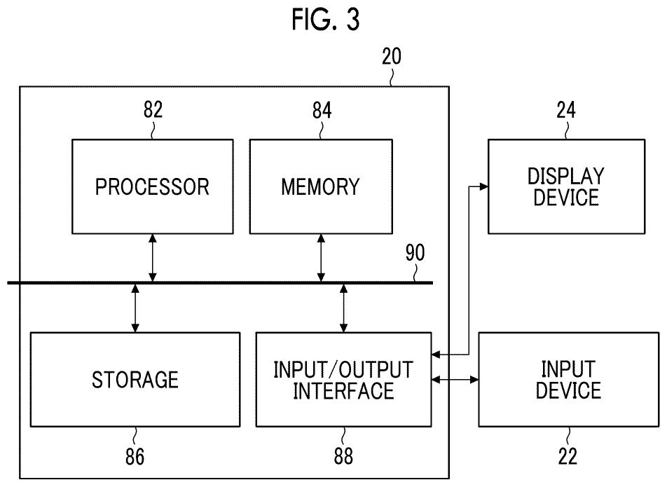

FIG. 3 is a block diagram showing a hardware configuration of an electric configuration of a console unit shown in FIG. 1. The examination control device 20 provided in the console unit 16 is configured using a computer. The computer applied to the examination control device 20 may be a personal computer, a workstation, or a server computer.

The processing function of the examination control device 20 may be implemented by a computer system including a plurality of computers. The computer applied to the console unit 16 may be a virtual machine.

The examination control device 20 comprises a processor 82, a memory 84 as a main memory, a storage 86 as an auxiliary memory, an input/output interface 88, and a bus 90.

The processor 82 includes one or more CPUs. The processor 82 may include a GPU. In addition, the examination control device 20 may further include a processor such as a DSP, an ASIC, and a PLD. Note that GPU is an abbreviation for Graphics Processing Unit, DSP is an abbreviation for Digital Signal Processor, ASIC is an abbreviation for Application Specific Integrated Circuit, and PLD is an abbreviation for Programmable Logic Device.

The processor 82 is connected to the memory 84, the storage 86, the input/output interface 88, the input device 22, and the display device 24 via the bus 90.

The memory 84 includes a RAM. The memory 84 may include a ROM. For example, the storage 86 may include a hard disk drive or may include a solid state drive. The storage 86 may be a combination of a hard disk drive and a solid state drive. The storage 86 may include an external storage device such as a removable disk.

Note that RAM is an abbreviation for Random Access Memory, and ROM is an abbreviation for Read Only Memory. The hard disk drive may be referred to as HDD using an abbreviation for Hard Disk Drive. The solid state drive may be referred to as an SSD using an abbreviation for Solid State Drive.

The storage device including the memory 84 and the storage 86 stores programs, data, and the like for implementing various functions of the MRI apparatus 10. The processor 82 executes the program stored in the memory 84 to implement various functions of the MRI apparatus 10. The processor 82 comprehensively controls each unit of the examination control device 20, various devices and units provided in the MRI apparatus 10, and the like to perform various kinds of processing.

The input/output interface 88 includes a communication interface that is connectable to a network, a connection interface that is connectable to an external device, and the like. As the input/output interface 88 that is connectable to the external device, for example, a universal serial bus or HDMI (registered trademark) can be applied. The universal serial bus may be referred to as USB using an abbreviation for Universal Serial Bus. HDMI is an abbreviation for High-Definition Multimedia Interface.

The processor 82 communicates with various devices of the MRI apparatus 10 via the input/output interface 88, thereby transmitting and receiving necessary information.

Various instructions and information input by the operator via the input device 22 are input to the examination control device 20. The operator interactively operates the MRI apparatus 10 by using the input device 22 and the display device 24.

The display device 24 displays various kinds of information in addition to the examination image captured by the MRI apparatus 10. The display device 24 is used as a part of a user interface in a case of receiving input from the input device 22. The present disclosure is not limited to one display device 24, and a form of a multi-display comprising a plurality of display devices can be used.

The hardware configuration of the electric configuration of the console unit 16 shown in FIG. 3 is various control devices provided in the console unit 16, and can be applied to various control devices to which a computer is applied.

The processor 82 described in the embodiment is an example of one or more processors that execute a program stored in one or more memories. In addition, the memory 84 described in the embodiment is an example of one or more memories that store a program including one or more instructions executed by the one or more processors.

### Electric Configuration of Examination Control Device

FIG. 4 is a functional block diagram showing an electric configuration of an examination control device shown in FIG. 2. The examination control device 20 comprises a measurement condition setting unit 100, a measurement control signal generation unit 102, and a measurement control signal output unit 104, as components related to the control of the measurement device 12 shown in FIG. 1. In addition, the examination control device 20 comprises a measurement data acquisition unit 106 and a measurement data processing unit 108 related to processing of the measurement result of the measurement device 12.

The measurement condition setting unit 100 sets a measurement condition to be applied to the sequence control device 48 shown in FIG. 2 based on an imaging protocol. Examples of the measurement condition include examinee identification information and an imaging part.

The measurement control signal generation unit 102 generates a measurement control signal to be applied to the sequence control device 48 based on the measurement condition. The measurement control signal output unit 104 transmits the measurement control signal generated by the measurement control signal generation unit 102 to the sequence control device 48. The sequence control device 48 controls the measurement of the examinee based on a specified imaging protocol.

The measurement data acquisition unit 106 acquires an NMR signal as measurement data of the examinee transmitted from the measurement device 12. The measurement data processing unit 108 generates a reconstructed image of the examinee using the acquired NMR signal of the examinee.

The examination control device 20 comprises a display signal generation unit 110. The display signal generation unit 110 generates a display signal representing information to be displayed on the display device 24 and transmits the display signal to the display device 24. For example, the display signal generation unit 110 generates a display signal representing the reconstructed image of the examinee generated by using the measurement data processing unit 108, and transmits the display signal representing the reconstructed image to the display device 24. The reconstructed image of the examinee is displayed on the display device 24.

The examination control device 20 comprises an examination table drive condition setting unit 120, an examination table control signal generation unit 122, an examination table control signal output unit 124, and a calibration data generation unit 126, as components related to the movement control of the tabletop 14A.

The examination table drive condition setting unit 120 sets a movement condition of the tabletop 14A on which the examinee is placed, based on an imaging protocol. Examples of the movement condition include identification information of the examinee and a measurement target part of the examinee.

The examination table control signal generation unit 122 generates a movement control signal for the tabletop 14A to be applied to the examination table drive unit 52. By the movement control signal for the tabletop 14A, a moving distance in the up-down direction of the tabletop 14A, a moving distance in the lateral direction of the tabletop 14A, and a moving distance in the front-rear direction of the tabletop 14A are specified. A movement speed of the tabletop 14A in each direction is appropriately specified.

The examination table control signal output unit 124 transmits a drive control signal to the examination table drive unit 52. The examination table drive unit 52 moves the tabletop 14A in at least any of the up-down direction, the lateral direction, or the front-rear direction based on the drive control signal.

The calibration data generation unit 126 generates calibration data for correcting the distance information transmitted from the camera 27. The calibration data is applied to the correction of the distance from the camera 27 to the examinee, which is derived by using the camera 27. Details of the calibration data generation will be described below.

The examination control device 20 comprises a camera image acquisition unit 130, a camera image processing unit 132, a distance information acquisition unit 134, a distance information processing unit 136, an actual measurement data acquisition unit 138, and an actual measurement data processing unit 140, as components related to the generation of the calibration data.

The camera image acquisition unit 130 acquires a camera image generated by using the camera 27 shown in FIG. 2. The camera image may be a live view image or a still image at any timing.

The camera image processing unit 132 performs specified processing on the camera image. For example, the camera image processing unit 132 performs processing of generating a guide or the like to be superimposed and displayed on the camera image.

The camera image processing unit 132 transmits data representing the camera image or the like to the display signal generation unit 110. The display signal generation unit 110 generates a display signal representing the camera image or the like and transmits the display signal to the display device 24. The camera image or the like is displayed on the display device 24.

A two-dimensional coordinate system is applied to the camera image, and the position in the camera image is specified using coordinate values. An example of the two-dimensional coordinate system applied to the camera image is a two-dimensional orthogonal coordinate system having two axes orthogonal to each other. An origin of the two-dimensional orthogonal coordinate system may be any of four corners of the camera image.

The distance information acquisition unit 134 acquires a distance from the camera 27 to the subject as the distance information transmitted from the camera 27.

The distance information processing unit 136 performs specified processing on the distance information acquired by using the distance information acquisition unit 134. The distance information processing unit 136 transmits the distance information that has been subjected to the specified processing to the display signal generation unit 110. The display signal generation unit 110 generates a display signal representing the distance information and transmits the display signal to the display device 24. The distance information is displayed on the display device 24. An example of the distance information is text information representing the distance from the camera 27 to the subject.

In addition, the distance information processing unit 136 transmits the distance information to the calibration data generation unit 126. The distance information is used for generating calibration data in the calibration data generation unit 126.

The actual measurement data acquisition unit 138 acquires actual measurement data measured by the operator. For example, the actual measurement data acquisition unit 138 acquires actual measurement data that is measured by the operator using a measurement instrument and that is input by the operator using the input device 22.

The actual measurement data processing unit 140 performs specified processing on the actual measurement data acquired by using the actual measurement data acquisition unit 138. The actual measurement data processing unit 140 transmits the actual measurement data to the calibration data generation unit 126. The actual measurement data is used for generating calibration data in the calibration data generation unit 126. The actual measurement data processing unit 140 may transmit the actual measurement data acquired by using the actual measurement data acquisition unit 138 to the display signal generation unit 110 and display the actual measurement data on the display device 24.

The calibration data generation unit 126, the camera image acquisition unit 130, the camera image processing unit 132, the distance information acquisition unit 134, the distance information processing unit 136, the actual measurement data acquisition unit 138, and the actual measurement data processing unit 140 shown in FIG. 4 can function as components of a calibration data generation device that generates calibration data applied to the correction of the movement parameter of the tabletop 14A.

Regarding Hardware Configuration of each Processing Unit

A hardware structure of a processing unit that executes various kinds of processing of the console unit 16 shown in FIG. 2 and the examination control device 20 shown in FIG. 4 is, for example, various processors as shown below.

Various processors include a CPU, which is a general-purpose processor that executes a program and functions as various processing units, GPU, a programmable logic device (PLD), which is a processor of which a circuit configuration can be changed after manufacture such as a field programmable gate array (FPGA), a dedicated electric circuit, which is a processor having a circuit configuration specially designed to execute specific processing such as an application specific integrated circuit (ASIC), and the like.

One processing unit may be configured of one of the various types of processors, or configured of a combination of two or more processors of the same type or different types. For example, one processing unit may be configured of a plurality of FPGAs, a combination of a CPU and an FPGA, or a combination of a CPU and a GPU. In addition, a plurality of processing units may be configured of one processor. As an example of configuring a plurality of processing units by one processor, first, as represented by a computer, such as a client or a server, there is a form in which one processor is configured by a combination of one or more CPUs and software and this processor functions as a plurality of processing units. Second, as represented by a system on chip (SoC), a processor that realizes the functions of the entire system including the plurality of processing units by using one integrated circuit (IC) chip is used. As described above, the various processing units are configured using one or more of the various processors as a hardware structure.

The hardware structure of these various processors is more specifically an electric circuit (circuitry) in which circuit elements such as semiconductor elements are combined.

### Generation of Up-Down Movement Calibration Data

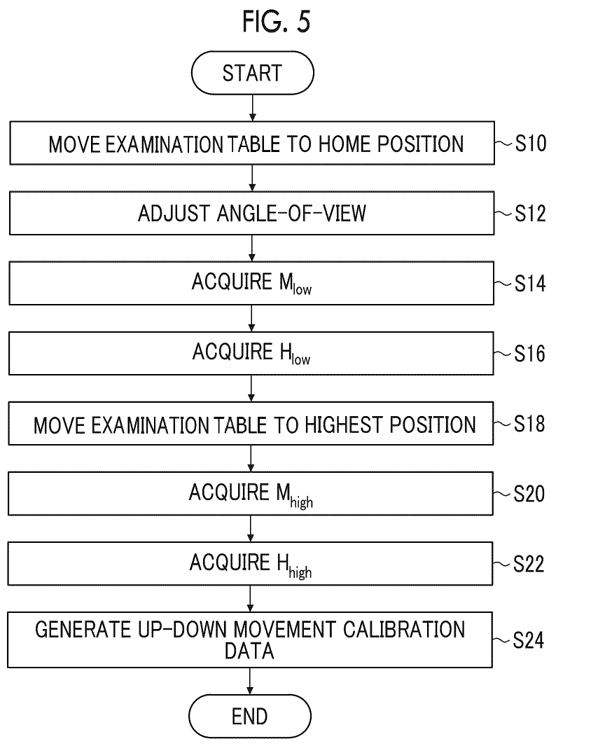

FIG. 5 is a flowchart showing a procedure of an up-down movement calibration data generation method. The movement of the tabletop 14A in the up-down direction represents the movement of the tabletop 14A in the up-down direction orthogonal to the installation surface PP of the examination table 14. For example, in a case in which the installation surface PP of the examination table 14 is a surface parallel to a horizontal plane, the up-down movement represents the movement of the tabletop 14A in a vertical upward direction and a vertical downward direction. The term “parallel” in the present specification is not limited to “strict parallel”, and may include “substantially parallel” that can be regarded as parallel. The term “orthogonal” may include “substantially orthogonal” as in the case of the “parallel”.

Hereinafter, for convenience of description, the lateral direction may be referred to as a DX direction, the front-rear direction may be referred to as a DY direction, and the up-down direction may be referred to as a DZ direction.

In generating the calibration data, a calibration data generation program is installed in advance in the calibration data generation unit 126, and a computer provided with a processor functioning as the calibration data generation unit 126 executes the calibration data generation program. The program is synonymous with software.

In a home position movement step S10, the examination control device 20 shown in FIG. 2 moves the tabletop 14A to a home position with respect to the examination table 14 installed at a position where the measurement of the examinee is started. The home position is a position where the tabletop 14A is at its lowest, and represents the lowest position of the tabletop 14A. After the home position movement step S10, the process proceeds to an angle-of-view adjustment step S12. The movement of the examination table 14 may be performed manually by the operator, or the examination table 14 may be operated automatically or semi-automatically.

In the angle-of-view adjustment step S12, the operator visually recognizes the camera image of the tabletop 14A, and a position of a headrest of the tabletop 14A is checked. That is, in the angle-of-view adjustment step S12, it is checked that the headrest is present at an upper end of a screen of the display device 24 in the camera image of the tabletop 14A. In a case of confirming the position of the headrest in the camera image, it may be checked that the gantry 11 is shown at an upper end of the camera image.

In a case in which the headrest is not shown within a specified range in the camera image of the examination table 14, an attachment direction of the camera unit 26 is adjusted, and the attachment direction of the camera unit 26 is changed to a specified attachment direction.

In addition, in the angle-of-view adjustment step S12, the operator visually recognizes the camera image of the tabletop 14A, and it is checked whether the examination table 14 is included in an examination table position guide frame displayed on a display screen of the camera image.

Further, in the angle-of-view adjustment step S12, the operator visually recognizes the camera image of the tabletop 14A, and it is checked whether all three marks on a straight guide displayed on the display screen of the camera image are displayed on the tabletop 14A.

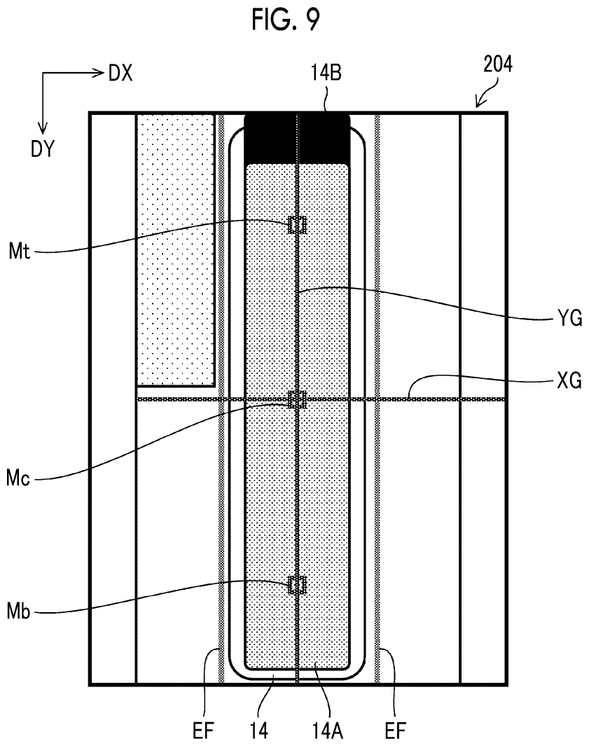

In a case in which the position of the examination table 14 does not satisfy the above condition, the position of the examination table 14 is adjusted to a position where the above condition is satisfied. Instead of adjusting the examination table 14, an attachment position of the camera 27 may be adjusted, or the adjustment of the examination table 14 and the adjustment of the attachment position of the camera 27 may be used in combination. In a case in which the position of the examination table 14 satisfies the above condition, the examination control device 20 acquires a signal representing the completion of the angle of view adjustment. The signal representing the completion of the angle-of-view adjustment may be transmitted to the examination control device 20 by the operator operating the input device 22. After the angle-of-view adjustment step S12, the process proceeds to a home position tabletop height acquisition step S14. The examination table position guide frame, the guide, and the mark are shown in FIG. 9.

In the home position tabletop height acquisition step S14, a home position tabletop height M_{low}, which is a height from the installation surface PP of the examination table 14 to an upper surface of the tabletop 14A at the home position, is acquired. The home position tabletop height M_{low} is a value measured by the operator, and a value input using the input device 22 can be acquired. After the home position tabletop height acquisition step S14, the process proceeds to a home position camera-to-tabletop distance acquisition step S16.

In the home position camera-to-tabletop distance acquisition step S16, a home position camera-to-tabletop distance H_{low}, which is a distance from the camera 27 to the tabletop 14A at the home position, is acquired. The distance information transmitted from the camera 27 may be applied to the home position camera-to-tabletop distance H_{low}. In a case in which the value of the distance information transmitted from the camera 27 is changed, the home position camera-to-tabletop distance H_{low} may be an approximate median value of the changed value. The operator may input the value of the distance information displayed on the display device 24 using the input device 22 as the home position camera-to-tabletop distance H_{low}.

The order of the home position tabletop height acquisition step S14 and the home position camera-to-tabletop distance acquisition step S16 may be interchanged, or both steps may be executed in parallel. After the home position camera-to-tabletop distance acquisition step S16, the process proceeds to a highest position movement step S18.

In the highest position movement step S18, the examination control device 20 raises the tabletop 14A from the home position and moves the tabletop 14A to the highest position. The movement of the tabletop 14A may be performed manually, automatically, or semi-automatically. After the highest position movement step S18, the process proceeds to a highest position tabletop height acquisition step S20.

In the highest position tabletop height acquisition step S20, a highest position tabletop height M_{high}, which is a height from the installation surface PP of the examination table 14 to the upper surface of the tabletop 14A at the highest position, is acquired. The highest position tabletop height M_{high} can be acquired in the same manner as the home position tabletop height M_{low}. After the highest position tabletop height acquisition step S20, the process proceeds to a highest position camera-to-tabletop distance acquisition step S22.

In the highest position camera-to-tabletop distance acquisition step S22, a highest position camera-to-tabletop distance H_{high}, which is a distance from the camera 27 to the tabletop 14A at the highest position, is acquired. The highest position camera-to-tabletop distance H_{high} is derived using the function of the camera 27 in the same manner as the home position camera-to-tabletop distance H_{low}. After the highest position camera-to-tabletop distance acquisition step S22, the process proceeds to an up-down movement calibration data generation step S24.

In the up-down movement calibration data generation step S24, the calibration data generation unit 126 generates up-down movement calibration data by using the home position tabletop height M_{low}, the home position camera-to-tabletop distance H_{low}, the highest position tabletop height M_{high}, and the highest position camera-to-tabletop distance H_{high}.

After the up-down movement calibration data is generated in the up-down movement calibration data generation step S24, the procedure of the up-down movement calibration data generation method is ended.

The home position of the tabletop 14A described in the embodiment is an example of a first up-down position, and the highest position of the tabletop 14A is an example of a second up-down position. The upper surface of the tabletop 14A at the home position described in the embodiment is an example of a first plane, the upper surface of the tabletop 14A at the highest position is an example of a second plane, and the upper surface of the tabletop 14A is an example of a placement surface.

The home position camera-to-tabletop distance H_{low} described in the embodiment is an example of first distance information, and the highest position camera-to-tabletop distance H_{high} is an example of second distance information. The camera image at the home position described in the embodiment is an example of first plane information, and the camera image at the highest position is an example of second plane information. The up-down movement calibration data described in the embodiment is an example of calibration data for correcting the movement parameter of the tabletop.

Specific Example of Examination table Height

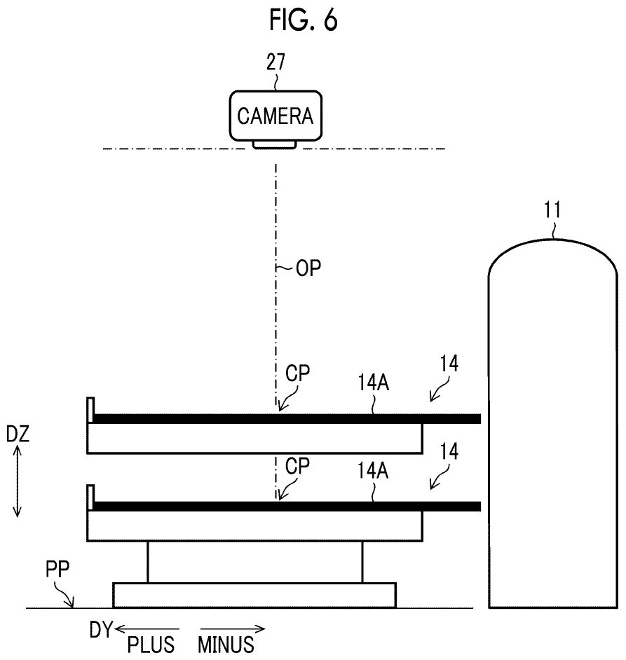

FIG. 6 is an explanatory diagram of an examination table height. In FIG. 6, the home position and the highest position of the tabletop 14A at a position of the examination table 14 where the tabletop 14A starts to enter the imaging space 11A of the gantry 11 are schematically shown. The upper tabletop 14A shown in FIG. 6 shows a state in which the upper tabletop 14A is stopped at the highest position. The lower tabletop 14A shown in FIG. 6 shows a state in which the lower tabletop 14A is stopped at the home position. The plus DY direction shown in FIG. 6 is a backward direction, and the minus DY direction is a forward direction.

The camera 27 shown in FIG. 6 is installed directly above a center position CP of the tabletop 14A at the home position, and the camera 27 is directed directly downward and is not inclined with respect to the horizontal plane. That is, an imaging optical axis OP of the camera 27 passes through the center position CP of the tabletop 14A and faces a direction orthogonal to the installation surface PP of the examination table 14.

However, in the actual imaging room 17A, the camera 27 may not be able to be installed directly above the center position CP of the tabletop 14A. For example, in a case in which an illumination device, a projector, or the like is installed directly above the center position CP of the tabletop 14A, the illumination device or the like interferes with the camera 27. In such a case, the camera 27 is installed to be shifted in at least one of the front-rear direction or the left-right direction.

### Specific Example of Camera Image Visually Recognized in Angle-of-View Adjustment Step

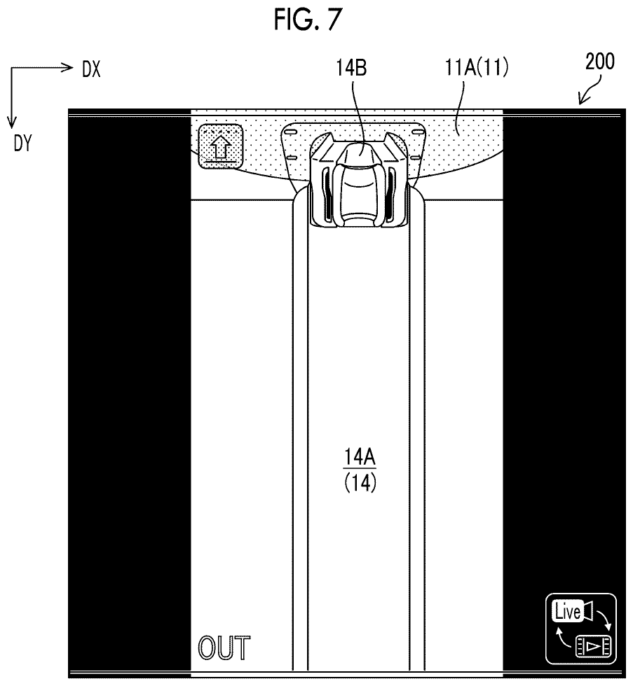

FIG. 7 is a schematic diagram of a screen on which a camera image is displayed. A camera image display screen 200 shown in FIG. 7 is a display screen for the camera image to be displayed on the display device 24. The camera image shown in FIG. 7 is a live view image.

In the angle-of-view adjustment step S12 shown in FIG. 5, the operator visually recognizes the camera image display screen 200 to check whether or not the gantry 11 and a headrest 14B are shown on an upper side of the camera image display screen 200.

In the camera image display screen 200 shown in FIG. 7, the imaging space 11A and the headrest 14B of the gantry 11 are shown on the upper side of the camera image display screen 200, and the adjustment of the installation direction of the camera 27 is not necessary in the angle-of-view adjustment step S12.

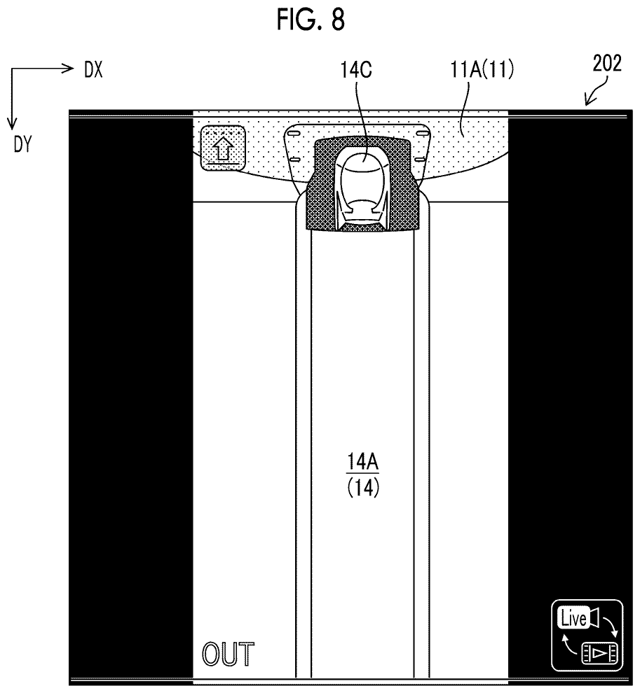

FIG. 8 is a schematic diagram of another example of a screen on which a camera image is displayed. A camera image shown on a camera image display screen 202 shown in FIG. 8 displays a headrest 14C having a different shape from the headrest 14B shown in FIG. 7. In the camera image display screen 202 shown in FIG. 8, the imaging space 11A of the gantry 11 and the headrest 14C are shown on an upper side of the camera image display screen 202, and the adjustment of the installation direction of the camera 27 is not necessary in the angle-of-view adjustment step S12.

### Specific Example of Guide Superimposed and Displayed on Camera Image

FIG. 9 is a schematic diagram showing a specific example of a guide displayed in a superimposed manner on a camera image. A camera image display screen 204 shown in FIG. 9 is displayed on the display device 24 in the angle-of-view adjustment step S12 shown in FIG. 5. The camera image display screen 204 displays a camera image in which the tabletop 14A of the examination table 14 is imaged, and an examination table position guide frame EF, a lateral guide XG, and a front-rear guide YG are superimposed and displayed on the camera image.

In addition, in the camera image display screen 204, a first mark Mt, a second mark Mc, and a third mark Mb, which represent the measurement position of the distance from the camera 27 to the tabletop 14A, are superimposed and displayed on the camera image by being superimposed on the front-rear guide YG.

The examination table position guide frame EF represents both ends in the lateral direction of a region in which the examination table 14 is disposed. In the angle-of-view adjustment step S12 shown in FIG. 5, the operator visually recognizes the camera image display screen 204 to adjust the position of the examination table 14 in the lateral direction, and disposes the examination table 14 between two examination table position guide frames EF. In a case in which the camera 27 is shifted in the left-right direction, the examination table position guide frame EF and the examination table 14 need only be parallel to each other in the left-right direction.

The lateral guide XG represents a center position in the front-rear direction in the camera image. The front-rear guide YG represents a center position in the lateral direction in the camera image. The second mark Mc is displayed at an intersection of the lateral guide XG and the front-rear guide YG. The second mark Mc represents a center position of the camera image and represents a measurement position at a distance from the camera 27 to the center position CP of the tabletop 14A.

In the angle-of-view adjustment step S12 shown in FIG. 5, the operator visually recognizes the camera image to check whether or not the first mark Mt, the second mark Mc, and the third mark Mb are superimposed and displayed on the tabletop 14A. In a case in which the first mark Mt, the second mark Mc, and the third mark Mb are not superimposed and displayed on the tabletop 14A, the position of the examination table 14 or the position of the camera 27 is adjusted, and the examination table 14 is disposed at a position where the first mark Mt, the second mark Mc, and the third mark Mb are superimposed and displayed on the tabletop 14A.

### Specific Example of Up-Down Movement Calibration Data

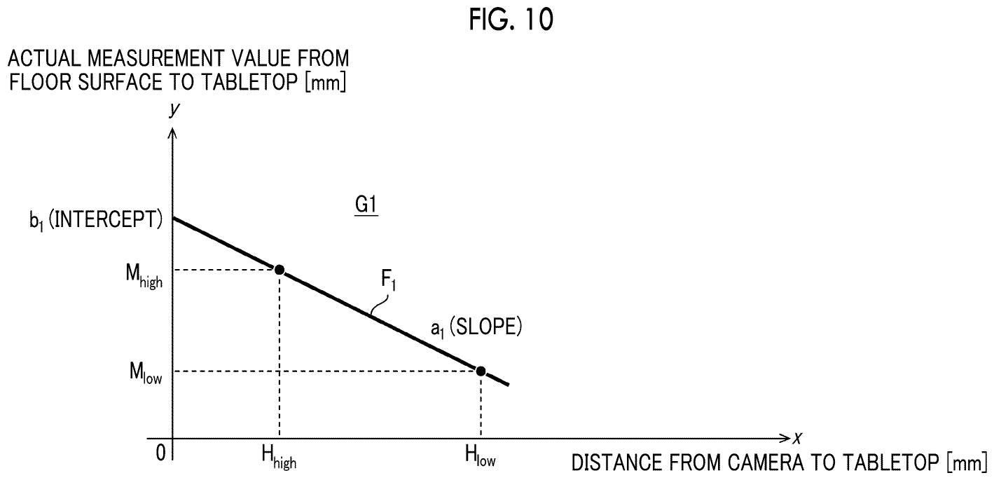

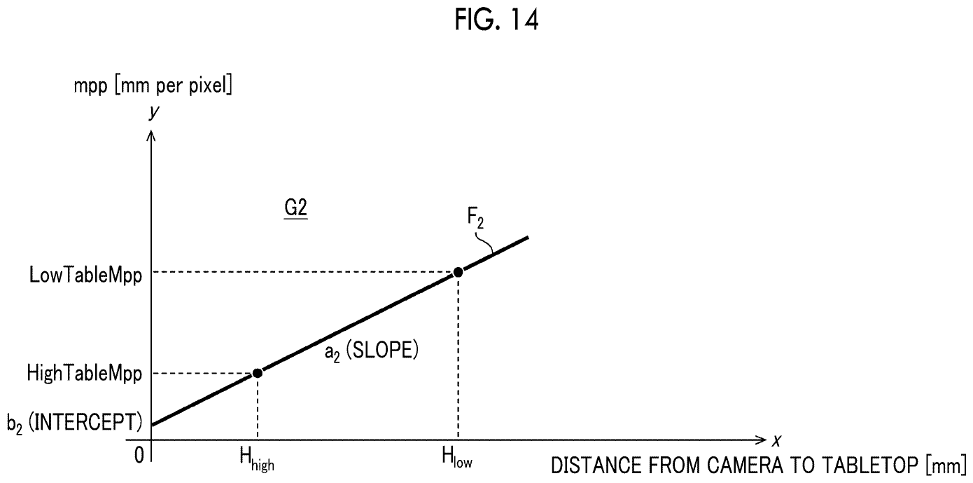

FIG. 10 is a graph showing an example of up-down movement calibration data. A horizontal axis of a graph G1 shown in FIG. 10 represents a distance from the camera 27 to the tabletop 14A. A vertical axis of the graph G1 represents an actual measurement value of a distance from the installation surface PP of the examination table 14 to the tabletop 14A. The unit of the distance from the camera 27 to the tabletop 14A and the unit of the distance from the installation surface PP of the examination table 14 to the tabletop 14A are millimeters. In addition, for convenience, in the graph G1, the horizontal axis is referred to as an x-axis, and the vertical axis is referred to as a y-axis. The same applies to a graph G2 shown in FIG. 14.

A linear function F₁ represented by the graph G1 is derived by using the home position tabletop height M_{low}, the home position camera-to-tabletop distance H_{low}, the highest position tabletop height M_{high}, and the highest position camera-to-tabletop distance H_{high}. The calibration data generation unit 126 shown in FIG. 4 derives a set of a value of a slope a₁ and a value of an intercept b₁ of the y-axis, which is a value axis, of the linear function F₁ as the up-down movement calibration data.

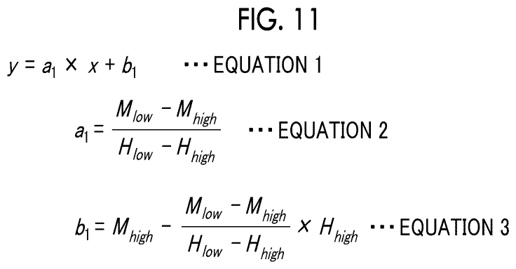

FIG. 11 is an explanatory diagram of a mathematical equation representing a linear function of the graph shown in FIG. 10. The linear function F₁ is represented by Equation 1 shown in FIG. 11. The slope a₁ used in Equation 1 is represented by Equation 2. In addition, the y-axis intercept b₁ used in Equation 1 is represented by Equation 3.

The linear function F₁ shown in FIGS. 10 and 11 is used for deriving a distance camera_iso_mm from the camera 27 to the ISO center in the up-down direction. For example, in a case in which the distance from the installation surface PP of the examination table 14 to the ISO center in the up-down direction is 1234 millimeters, in the linear function F₁, a value of x in a case in which y=1234 is the distance camera_iso_mm from the camera 27 to the ISO center in the up-down direction. That is, the linear function F₁ is represented by x=(y−b₁)/a₁, and camera_iso_mm=(1234−b₁)/a₁.

The calibration data is applied to the derivation of a movement amount of the tabletop 14A in the up-down direction. A distance camera_patient_mm from the camera 27 to the examinee is obtained using the camera 27. A movement amount move_height_mm of the tabletop 14A in the up-down direction is calculated by subtracting the distance camera_iso_mm from the camera 27 to the ISO center from the distance camera_patient_mm from the camera 27 to the examinee.

That is, the movement amount move_height_mm of the tabletop 14A in the up-down direction is represented by (move_height_mm)=(camera_iso_mm)−(camera_point_mm).

### Specific Example of Generating Front-Rear Lateral Movement Calibration Data

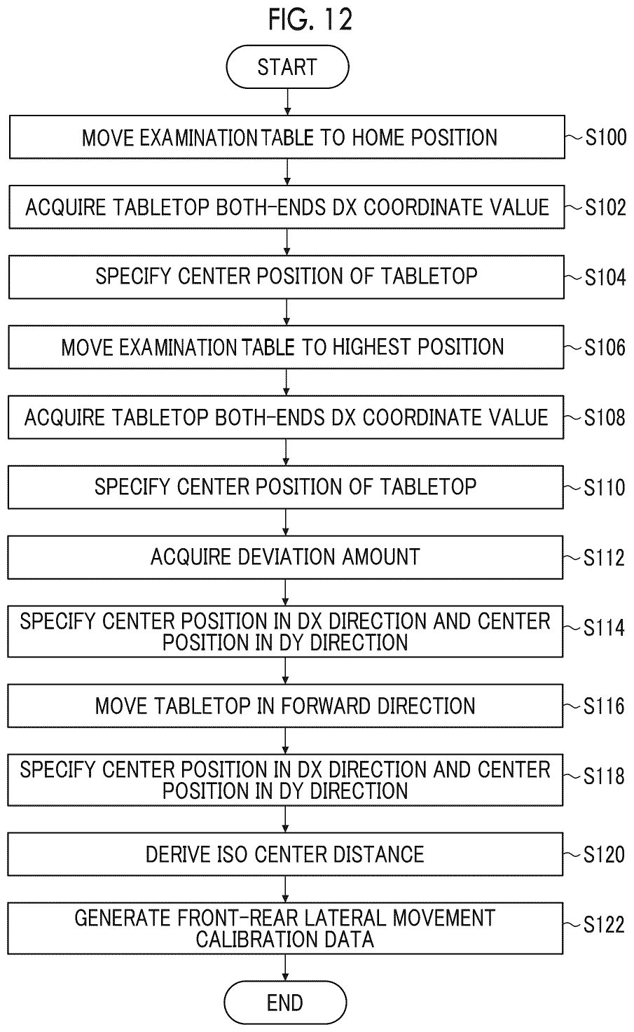

FIG. 12 is a flowchart showing a procedure of a front-rear lateral movement calibration data generation method. In a home position movement step S100, the examination control device 20 shown in FIG. 2 moves the tabletop 14A to the home position, similarly to the home position movement step S10 shown in FIG. 5. After the home position movement step S100, the process proceeds to a home position tabletop both-ends coordinate acquisition step S102.

In the home position tabletop both-ends coordinate acquisition step S102, the examination control device 20 acquires coordinates of both ends of the tabletop 14A in the lateral direction. Specifically, a two-dimensional orthogonal coordinate system that is specified in the camera image and that is based on a DX axis along the lateral direction and a DY axis along the front-rear direction is applied to acquire a coordinate value LowTableLeftPointX of one end of the tabletop 14A in the DX direction and a coordinate value LowTableRightPointX of the other end. The coordinate values are represented by the number of pixels.

In the home position tabletop both-ends coordinate acquisition step S102, the operator may operate a mouse to click both ends of the tabletop 14A in the DX direction in the camera image as a mouse event to acquire the coordinate values of both ends of the tabletop 14A in the DX direction. After the home position tabletop both-ends coordinate acquisition step S102, the process proceeds to a home position tabletop center position specification step S104.

In the home position tabletop center position specification step S104, the position of the tabletop 14A overlapping the center of the camera image is marked using the front-rear guide YG and the lateral guide XG superimposed and displayed on the camera image as markers. After the home position tabletop center position specification step S104, the process proceeds to a highest position movement step S106.

In the highest position movement step S106, the examination control device 20 raises the tabletop 14A from the home position and moves the tabletop 14A to the highest position, similarly to the highest position movement step S18 shown in FIG. 5. After the highest position movement step S106, the process proceeds to a highest position tabletop both-ends coordinate acquisition step S108.

In the highest position tabletop both-ends coordinate acquisition step S108, the examination control device 20 acquires a coordinate value HighTableLeftPointX of one end of the tabletop 14A in the DX direction at the highest position and a coordinate value HighTableRightPointX of the other end, similarly to the home position tabletop both-ends coordinate acquisition step S102. After the highest position tabletop both-ends coordinate acquisition step S108, the process proceeds to a highest position tabletop center position specification step S110.

In the highest position tabletop center position specification step S110, the position of the tabletop 14A overlapping the center of the camera image is marked using the front-rear guide YG and the lateral guide XG superimposed and displayed on the camera image as markers, similarly to the home position tabletop center position specification step S104. After the highest position tabletop center position specification step S110, the process proceeds to a deviation amount acquisition step S112.

In the deviation amount acquisition step S112, the operator measures a distance between the marking at the home position and the marking at the highest position in the DX direction and the DY direction.

That is, in the deviation amount acquisition step S112, a DX deviation amount UpTableZureX representing the deviation amount of the center position of the camera image due to the up-and-down movement of the tabletop 14A in the DX direction and a DY deviation amount UpTableZureY representing the deviation amount of the center position of the camera image due to the up-and-down movement of the tabletop 14A in the DY direction are acquired. After the deviation amount acquisition step S112, the process proceeds to a pre-forward movement center position specification step S114.

In the pre-forward movement center position specification step S114, the center position in the DX direction of the tabletop 14A and the center position in the DY direction of the tabletop 14A in the camera image are specified at a pre-forward movement position. That is, in the pre-forward movement center position specification step S114, the center position in the DX direction of the tabletop 14A in the camera image is marked and the center position in the DY direction of the tabletop 14A in the camera image is marked, by using the lateral guide XG and the front-rear guide YG superimposed and displayed on the camera image as markers. After the pre-forward movement center position specification step S114, the process proceeds to a forward movement step S116.

In the forward movement step S116, the tabletop 14A is moved in the forward direction to a position where the tabletop 14A hits a light localizer indicating the ISO center. After the forward movement step S116, the process proceeds to a post-forward movement center position specification step S118.

In the post-forward movement center position specification step S118, the center position in the DX direction of the tabletop 14A and the center position in the DY direction of the tabletop 14A in the camera image are specified at a post-forward movement position, similarly to the pre-forward movement center position specification step S114. After the post-forward movement center position specification step S118, the process proceeds to an ISO center distance derivation step S120.

In the ISO center distance derivation step S120, an ISO center distance ISOcenterDistanceX in the DX direction is derived based on the center position in the DX direction of the tabletop 14A specified in the pre-forward movement center position specification step S114 and the center position in the DX direction of the tabletop 14A specified in the post-forward movement center position specification step S118.

In addition, in the ISO center distance derivation step S120, an ISO center distance ISOcenterDistanceY in the DY direction is derived based on the center position in the DY direction of the tabletop 14A specified in the pre-forward movement center position specification step S114 and the center position in the DY direction of the tabletop 14A specified in the post-forward movement center position specification step S118. After the ISO center distance derivation step S120, the process proceeds to a front-rear lateral movement calibration data generation step S122.

In the front-rear lateral movement calibration data generation step S122, the calibration data generation unit 126 calculates a home position length-pixel conversion value LowTableMpp for converting the number of pixels at the home position into a length, based on the coordinate value LowTableLeftPointX of one end in the DX axis of the tabletop 14A at the home position and the coordinate value LowTableRightPointX of the other end.

In addition, in the front-rear lateral movement calibration data generation step S122, the calibration data generation unit 126 calculates a highest position length-pixel conversion value HighTableMpp for converting the number of pixels at the highest position into a length by using the coordinate value HighTableLeftPointX of one end in the DX axis of the tabletop 14A at the highest position and the coordinate value HighTableRightPointX of the other end.

Further, in the front-rear lateral movement calibration data generation step S122, the calibration data generation unit 126 generates front-rear lateral movement calibration data by using the home position camera-to-tabletop distance H_{low} representing the distance from the camera 27 to the tabletop 14A at the home position and the highest position camera-to-tabletop distance H_{high} representing the distance from the camera 27 to the tabletop 14A at the highest position.

After the front-rear lateral movement calibration data is generated in the front-rear lateral movement calibration data generation step S122, the procedure of the front-rear lateral movement calibration data generation method is ended.

### Specific Example of Tabletop Both-Ends Coordinate Acquisition

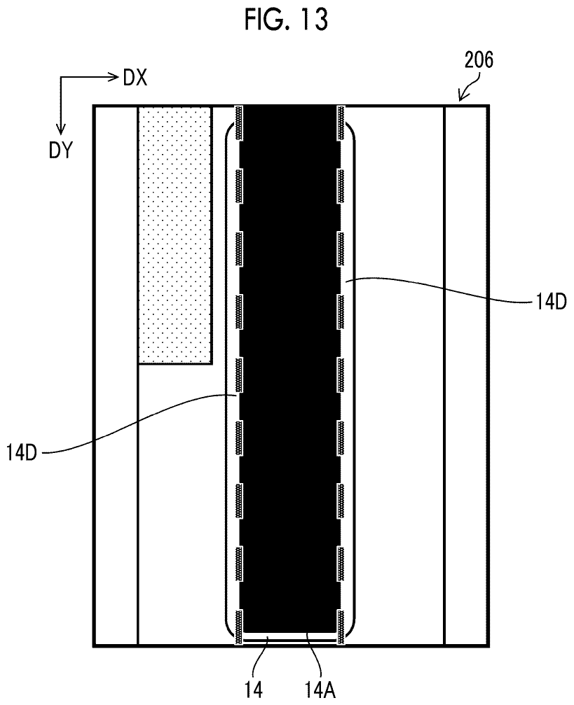

FIG. 13 is an explanatory diagram of tabletop both-ends coordinate acquisition. In FIG. 13, a display screen 206 of the camera image is schematically shown. A guide for the tabletop 14A in the DX direction may be a boundary between a side cover 14D and the tabletop 14A, which is shown using a broken line. A left end of the tabletop 14A in FIG. 13 corresponds to the coordinate value LowTableLeftPointX and the coordinate value HighTableLeftPointX. In addition, a right end of the tabletop 14A corresponds to the coordinate value LowTableRightPointX and the coordinate value HighTableRightPointX.

In a case in which a total width of the tabletop 14A in the DX direction is denoted by H_{T}, the home position length-pixel conversion value LowTableMpp is represented by (LowTableMpp)=H_{T}/{(LowTableRightPointX)−(LowTableLeftPointX)}. The coordinate value LowTableRightPointX has a value exceeding a value of the coordinate value LowTableLeftPointX.

In addition, the highest position length-pixel conversion value HighTableMpp is represented by (HighTableMpp)=H_{T}/{(HighTableRightPointX)−(HighTableLeftPointX)}. The coordinate value HighTableRightPointX has a value exceeding a value of the coordinate value HighTableLeftPointX.

### Specific Example of Front-Rear Lateral Movement Calibration Data

FIG. 14 is a graph showing an example of front-rear lateral movement calibration data. A horizontal axis of a graph G2 shown in FIG. 14 represents a distance from the camera 27 to the tabletop 14A. The unit of the horizontal axis of the graph G2 is millimeters. A vertical axis of the graph G2 represents a distance mpp on the tabletop 14A per pixel of the camera image. The unit of the vertical axis of the graph G2 is millimeters per pixel.

A linear function F₂ represented by the graph G2 is derived by using the home position camera-to-tabletop distance H_{low}, the highest position camera-to-tabletop distance H_{high}, the home position length-pixel conversion value LowTableMpp, and the highest position length-pixel conversion value HighTableMpp. The calibration data generation unit 126 shown in FIG. 4 derives a set of a value of a slope a₂ and a value of an intercept b₂ of the y-axis, which is a value axis, of the linear function F₂ as the front-rear lateral movement calibration data.

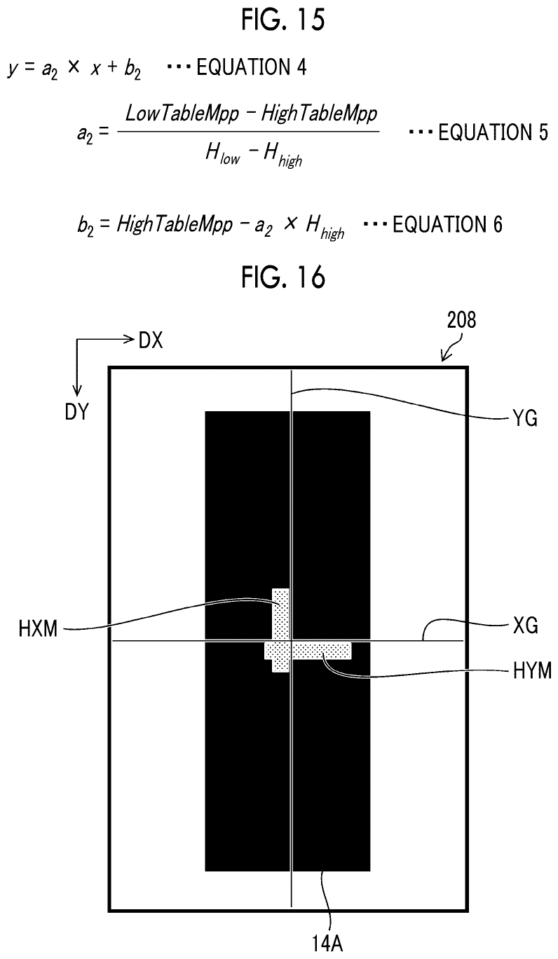

FIG. 15 is an explanatory diagram of a mathematical equation representing a linear function of the graph shown in FIG. 14. The linear function F₂ shown by a graph G2 in FIG. 14 is represented by Equation 4 shown in FIG. 15. The slope a₂ used in Equation 4 is represented by Equation 5. In addition, the y-axis intercept b₂ used in Equation 4 is represented by Equation 6.

### Specific Example of Marking at Home Position

FIG. 16 is an explanatory diagram of an example of marking of a center position at the home position. In a camera image display screen 208 shown in FIG. 16, a home position DX marking HXM and a home position DY marking HYM applied to the tabletop 14A in the home position tabletop center position specification step S104 shown in FIG. 12 are shown.

The home position DX marking HXM is located at a position along the front-rear guide YG and is provided at a position intersecting the lateral guide XG. The home position DY marking HYM is located at a position along the lateral guide XG and is provided at a position intersecting the front-rear guide YG.

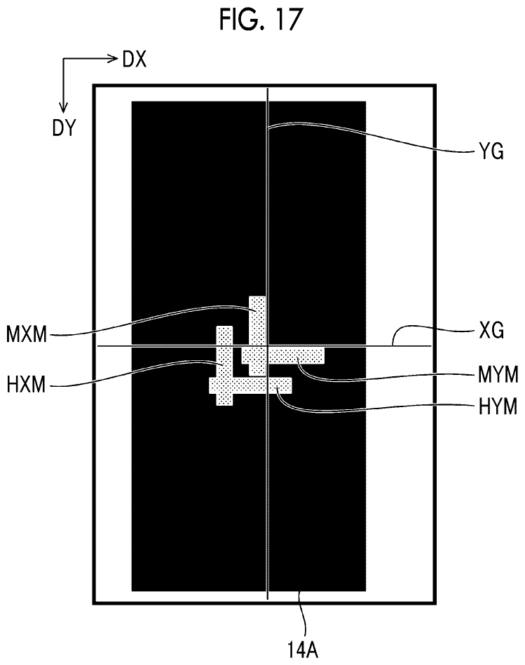

FIG. 17 is an explanatory diagram of an example of marking of a camera center at the highest position. FIG. 17 shows a highest position DX marking MXM and a highest position DY marking MYM applied to the tabletop 14A in the highest position tabletop center position specification step S110 shown in FIG. 12.

The highest position DX marking MXM is located at a position along the front-rear guide YG and is provided at a position intersecting the lateral guide XG. The highest position DY marking MYM is located at a position along the lateral guide XG and is provided at a position intersecting the front-rear guide YG.

### Specific Example of Acquisition of Acquisition of Deviation Amount of Tabletop Center Position

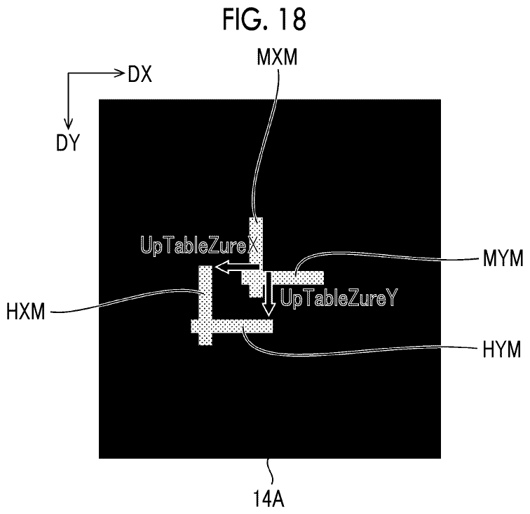

FIG. 18 is an explanatory diagram of acquisition of a deviation amount of tabletop center position. FIG. 18 shows the home position DX marking HXM, the home position DY marking HYM, the highest position DX marking MXM, and the highest position DY marking MYM shown in FIG. 17 in an enlarged manner.

In the deviation amount acquisition step S112 shown in FIG. 12, the operator measures a distance between the home position DX marking HXM and the highest position DX marking MXM in the DX direction, and derives a DX deviation amount UpTableZureX. The unit of the DX deviation amount UpTableZureX is millimeters.

In a case in which the home position DX marking HXM is positioned on the right side with respect to the highest position DX marking MXM, the DX deviation amount Up TableZureX is set to a value of minus. In addition, in a case in which the home position DX marking HXM is positioned on the left side with respect to the highest position DX marking MXM, the DX deviation amount UpTableZureX is set to a value of plus.

FIG. 18 shows an aspect in which a distance between the markings is measured based on a right end of each marking as a reference, but a left end of each marking may be used as a reference, or a center of each marking may be used as a reference.

In addition, in the deviation amount acquisition step S112, the operator measures a distance between the home position DY marking HYM and the highest position DY marking MYM in the DY direction, and derives a DY deviation amount UpTableZureY. The unit of the DY deviation amount UpTableZureY is millimeters.

In a case in which the home position DY marking HYM is positioned on the upper side with respect to the highest position DY marking MYM, the DY deviation amount UpTableZureY is set to a value of minus. In addition, in a case in which the home position DY marking HYM is positioned on the lower side with respect to the highest position DY marking MYM, the DY deviation amount UpTableZureY is set to a value of plus.

FIG. 18 shows an aspect in which a distance between the markings is measured based on an upper end of each marking as a reference, but a lower end of each marking may be used as a reference, or a center of each marking may be used as a reference.

### Specific Example of Marking in Post-Forward Movement Center Position Specification Step

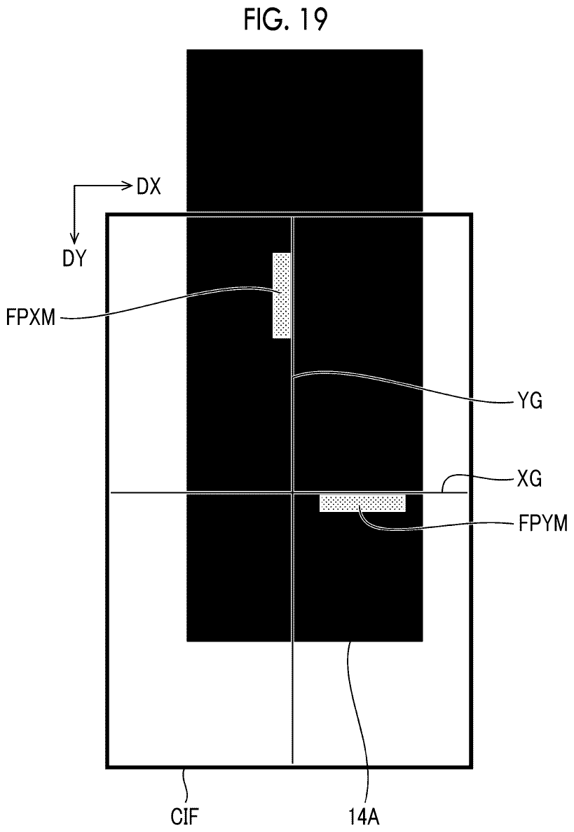

FIG. 19 is an explanatory diagram of an example of marking after the tabletop is moved forward. In FIG. 19, a frame CIF representing a display range of the camera image, a lateral guide XG superimposed and displayed on the camera image, and a front-rear guide YG are shown.

In the post-forward movement center position specification step S118 shown in FIG. 12, the operator applies a forward movement position X marking FPXM along the front-rear guide YG and applies a forward movement position Y marking FPYM along the lateral guide XG to the tabletop 14A.

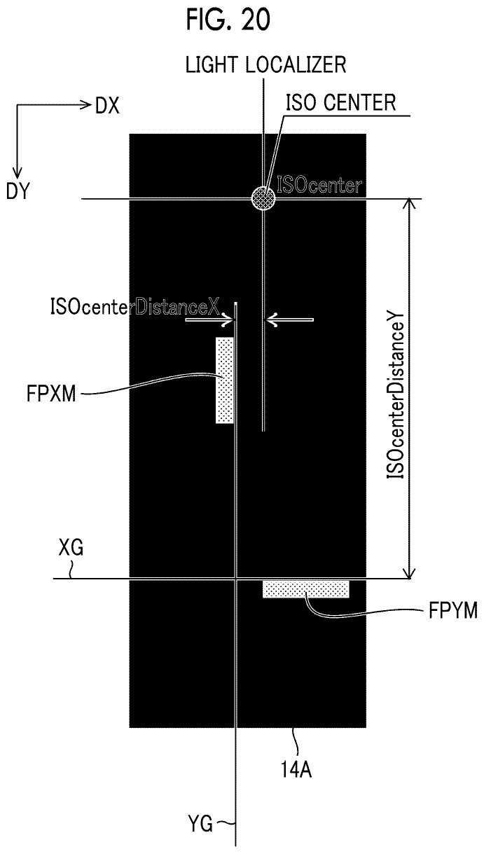

FIG. 20 is an explanatory diagram of an example of a difference value between a camera center and an ISO center. FIG. 20 shows a DX difference value ISOcenterDistanceX, which is a difference value between the camera center and the ISO center in the lateral direction, and a DY difference value ISOcenterDistanceY, which is a difference value between the camera center and the ISO center in the front-rear direction.

A light localizer shown in FIG. 20 is a ray representing the ISO center, and is used as a marker for alignment between the examinee and the center of imaging. A laser beam or the like is applied as the light localizer.

The operator measures the DX difference value ISOCenterDistanceX and the DY difference value ISOCenterDistanceY, and inputs the measured values to the examination control device 20. In a case in which the camera 27 is attached directly above a position of the ISO center in the lateral direction, a measured value of the DX difference value ISOcenterDistanceX is theoretically zero. The units of the DX difference value ISOCenterDistanceX and the DY difference value ISOCenterDistanceY are millimeters.

### Specific Example of Calibration in Lateral Direction and Front-Rear Direction

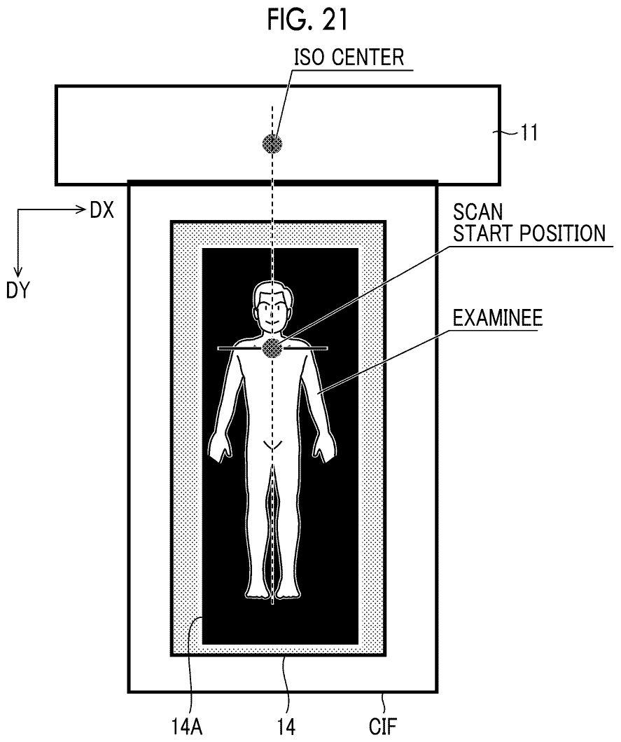

FIG. 21 is a schematic diagram showing a specific example of calibration in a lateral direction and a front-rear direction. FIG. 21 schematically shows a camera image of the examinee placed on the tabletop 14A of the examination table 14.

The alignment between a scan start position of the examinee and the ISO center, in which the calibration data is used, is performed according to the following procedure. First, coordinate values (middle_line, middle_line_st) of the scan start position in a front-rear lateral plane are estimated from the camera image. Here, an origin of a two-dimensional orthogonal coordinate system applied to the camera image is set to an upper left end of the camera image. The number of pixels from the origin is applied as the coordinate value.

Next, the value of the slope a₂ and the value of the y-axis intercept b₂ of the linear function F₂ shown in FIG. 14 are read. An estimated value H_ₑₛₜᵢₘₐₜₑ of the distance from the camera 27 to the tabletop 14A at a height of the tabletop 14A at which the scan start position is estimated is substituted for x in Equation 4 representing the linear function F₂ shown in FIG. 15, and a length-pixel conversion value Estimate_TableMpp at the height of the tabletop 14A at which the scan start position is estimated is calculated. That is, the length-pixel conversion value Estimate_TableMpp is represented by (Estimate_TableMpp)=a₂×(H_ₑₛₜᵢₘₐₜₑ)+b₂.

Next, the number of pixels from the center position of the camera image to the scan start position is converted into a distance in a unit such as millimeters, by using the length-pixel conversion value Estimate_TableMpp. The center position of the camera image is an intersection of the lateral guide XG and the front-rear guide YG shown in FIG. 20.

A distance middle_line_mm from the center position of the camera image in the lateral direction to the scan start position is represented by (middle_line_mm)={PNX−(middle_line)}×(Estimate_TableMpp). PNX is a half value of the number of pixels in the lateral direction of the camera image. In a case in which the number of pixels in the lateral direction of the camera image is 600, PNX is 300.

In addition, a distance middle_line_st_mm from the center position of the camera image in the front-rear direction to the scan start position is represented by (middle_line_st_mm)={(middle_line_st)−PNY}×Estimate_TableMpp. PNY is a half value of the number of pixels in the front-rear direction of the camera image. In a case in which the number of pixels in the front-rear direction of the camera image is 1200, PNY is 600.

The distance from the center position of the camera image to the scan start position is a value estimated at the height of the tabletop 14A at which the scan start position is estimated. This value is converted into a value at the highest position of the tabletop 14A.

First, a distance high_table_middle_line_mm from the center position of the camera image in the lateral direction to the scan start position at the highest position is represented by (high_table_middle_line_mm)=(middle_line_mm)+{(H_ₑₛₜᵢₘₐₜₑ−H_{high})/(H_{low}−H_{high})}×(UpTableZureX).

In addition, a distance high_table_middle_line_st_mm from the center position of the camera image in the front-rear direction to the scan start position at the highest position is represented by (high_table_middle_line_st_mm)=(middle_line_st_mm)+{(H_ₑₛₜᵢₘₐₜₑ−H_{high})/(H_{low}−H_{high})}×(UpTableZureY). The scan start position described in the embodiment is an example of a measurement start position specified for the examinee.

Next, a moving distance move_shift_mm of the tabletop 14A in the lateral direction and a moving distance move_position_mm of the tabletop 14A in the front-rear direction are calculated. The moving distance move_shift_mm of the tabletop 14A in the lateral direction is represented by (move_shift_mm)=(ISOcenterDistanceX)−(high_table_middle_line_mm).

In addition, the moving distance move_position_mm of the tabletop 14A in the front-rear direction is represented by (move_position_mm)=(ISOcenterDistanceY)−(high_table_middle_line_st_mm).

In a case in which the value of the moving distance move_shift_mm of the tabletop 14A in the lateral direction is minus, the tabletop 14A is moved in a right direction in FIG. 21. On the other hand, in a case in which the value of the moving distance move_shift_mm is plus, the tabletop 14A is moved in a left direction in FIG. 21.

The moving distance move_position_mm of the tabletop 14A in the front-rear direction is always in the minus direction, and the tabletop 14A is moved in the forward direction.

Specific Example of Calibration in Case in which Camera is Installed in Inclined State

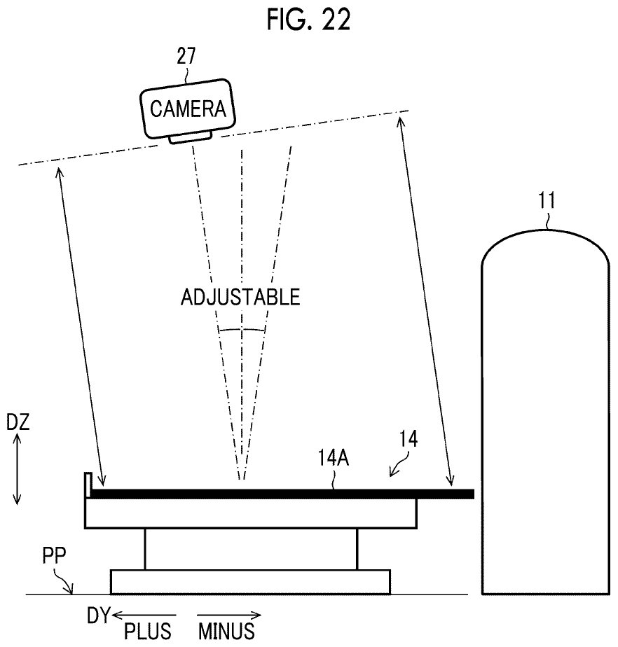

FIG. 22 is a schematic diagram of a distance from a camera to the tabletop in a case in which the camera is inclined with respect to an installation surface. The camera 27 may be inclined with respect to the installation surface PP so that the tabletop 14A is included in a frame of a camera image. For example, the imaging optical axis OP of the camera 27 is rotated about a rotation axis parallel to the lateral direction, and the inclination of the camera 27 is adjusted in a range of, for example, plus 5 degrees to minus 5 degrees.

In a case in which the camera 27 is inclined and adjusted, the distance from the camera 27 to the tabletop 14A varies depending on the position of the tabletop 14A in the front-rear direction. Therefore, in the adjustment of the attachment position in the front-rear direction and in the lateral direction, the distance from the camera 27 to the tabletop 14A for each position of the tabletop 14A in the front-rear direction is adopted.

That is, the distance from the camera 27 to the tabletop 14A affects the accuracy in calculating the moving distance of the tabletop 14A in the front-rear direction. Therefore, it is necessary to adjust the attachment direction of the camera 27 in which the inclination of the camera 27 with respect to the installation surface PP is considered.

In a case in which an orientation of the camera 27 is adjusted so that the imaging optical axis OP of the camera 27 is orthogonal to the installation surface PP, a distance from the camera 27 to a center position of the camera image shown in the camera image is measured as the distance from the camera 27 to the tabletop 14A. That is, the linear function F₁ shown in FIG. 10 and the linear function F₂ shown in FIG. 14 are based on the center position of the camera image.

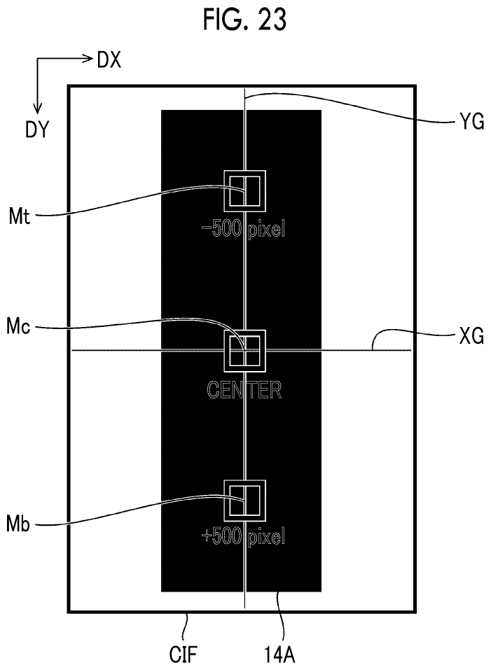

In a case in which the orientation of the camera 27 with respect to the installation surface PP is adjusted to be inclined within a specified range, a distance from the camera 27 to the tabletop 14A measured at a position different from the center position of the camera image is converted into a value at the center position of the camera image. A linear function used for this conversion is prepared. A first mark Mt, a second mark Mc, and a third mark Mb shown in FIG. 23 are used for deriving the linear function.

At the home position of the examination table 14, a distance from the camera 27 to the second mark Mc is denoted by D_center, a distance from the camera 27 to the first mark Mt is denoted by D_minus, and a distance from the camera 27 to the third mark Mb is denoted by D_plus. Each of the above distances is acquired as distance information of the camera 27. The unit of each distance is millimeters.

A linear function F₃ on a minus side in the front-rear direction is represented by z=a₃×y. Each of z and y in the mathematical equation representing the linear function F₃ represents a value in the up-down direction and a value in the front-rear direction. The same applies to the following linear function F₄.

In a case in which the number of pixels representing a distance from the second mark Mc to the first mark Mt in the front-rear direction is set to −500 pixels, a slope a₃ of the linear function F₃ is represented by a₃={(D_center)−(D_minus)}/{0−(−500)}={(D_center)−(D_minus)}/500.

Similarly, the linear function F₄ on a plus side in the front-rear direction is represented by z=a₄×y. In a case in which the number of pixels representing a distance from the second mark Mc to the third mark Mb in the front-rear direction is set to +500 pixels, a slope a₄ of the linear function F₄ is represented by a₄={(D_center)−(D_plus)}/{0−(+500)}={(D_center)−(D_plus)}/(−500). Here, 0 in the mathematical equation representing the slope a₃ of the linear function F₃ and in the mathematical equation representing the slope a₄ of the linear function F₄ means the coordinate value of the center position of the camera image.

That is, the number of pixels from the second mark Mc to the first mark Mt and the number of pixels from the second mark Mc to the third mark Mb represent that the second mark Mc is used as a reference. The slope a₃ of the linear function F₃ and the slope a₄ of the linear function F₄ are stored as calibration data.

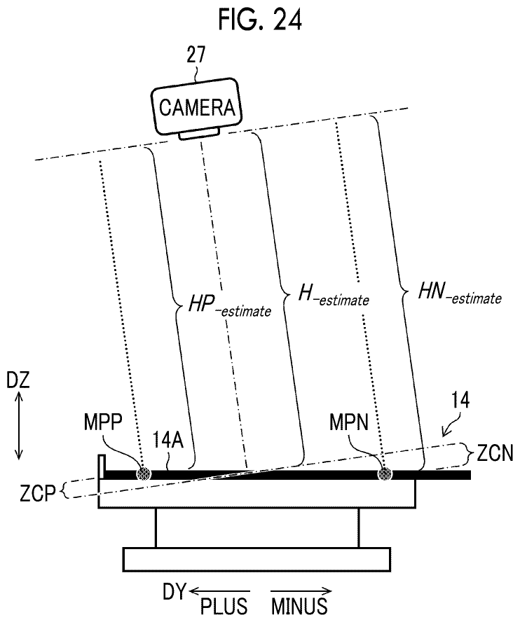

FIG. 24 is a schematic diagram of calibration in a case in which the camera is inclined with respect to the installation surface. In a case in which the camera is installed in a state of being inclined with respect to the installation surface PP, a correction value with respect to the estimated value H_ₑₛₜᵢₘₐₜₑ of the distance from the camera 27 to the tabletop 14A is calculated.

In the calculation of the correction value, a coordinate value Yₑₛₜᵢₘₐₜₑ in the front-rear direction of a measurement position on the tabletop 14A in a case in which the estimated value H_ₑₛₜᵢₘₐₜₑ of the distance from the camera 27 to the tabletop 14A is estimated is calculated. In a case in which an estimated position MPN on the minus side in the front-rear direction is specified and the coordinate value Yₑₛₜᵢₘₐₜₑ is a value of minus, the coordinate value Yₑₛₜᵢₘₐₜₑ is substituted for y of the linear function F₃, and a correction value ZCN is calculated as the value of z.

An estimated value HN_ₑₛₜᵢₘₐₜₑ of the distance from the camera 27 on the minus side in the front-rear direction to the tabletop 14A is calculated as (HN_ₑₛₜᵢₘₐₜₑ)=(H_ₑₛₜᵢₘₐₜₑ)+(ZCN) by using the estimated value H_ₑₛₜᵢₘₐₜₑ and the correction value ZCN.

Similarly, in a case in which a measurement position MPP on the plus side in the front-rear direction is specified and the coordinate value Yₑₛₜᵢₘₐₜₑ is a value of plus, the coordinate value Yₑₛₜᵢₘₐₜₑ is substituted for y of the linear function F₄, and a correction value ZCP is calculated as the value of z.

An estimated value HP_ₑₛₜᵢₘₐₜₑ of the distance from the camera 27 on the plus side in the front-rear direction to the tabletop 14A is calculated as (HP_ₑₛₜᵢₘₐₜₑ)=(H_ₑₛₜᵢₘₐₜₑ)−(ZCP) by using the measured value H_ₑₛₜᵢₘₐₜₑ and the correction value ZCP.

### Effects of Embodiment

The examination control device 20 according to the embodiment can obtain the following effects.

[1]

The camera 27 installed on the ceiling of the imaging room 17A is adjusted in position and posture such that the entire tabletop 14A is shown in the camera image and the headrest 14B and the like are reflected at a specified position in the camera image.

At the home position of the tabletop 14A provided in the examination table 14, the home position camera-to-tabletop distance H_{low}, which is the distance from the camera 27 to the tabletop 14A of the examination table 14, is measured using the camera 27 installed on the ceiling of the imaging room 17A, and the operator measures the home position tabletop height M_{low}, which is a height from the installation surface PP of the examination table 14 to the upper surface of the tabletop 14A.

At the highest position where the tabletop 14A is at its highest, the highest position camera-to-tabletop distance H_{high} is measured as a distance from the camera 27 to the tabletop 14A of the examination table 14, and the operator measures the highest position tabletop height M_{high}, which is a height from the installation surface PP of the examination table 14 to the upper surface of the tabletop 14A.

From the measurement results, the linear function F₁ representing a relationship between the distance from the camera 27 to the tabletop 14A and the distance from the installation surface PP to the tabletop 14A is derived. A combination of the slope a₁ and the y-axis intercept bi of the linear function F₁ is derived and stored as the up-down direction calibration data.

As a result, even in a case in which the position where the camera 27 is installed is shifted from a position specified in advance, it is possible to correct the moving distance of the tabletop 14A in the up-down direction based on the measurement result of the distance from the camera 27 to the examinee.

[2]

At the home position of the tabletop 14A, the number of pixels corresponding to the entire length of the tabletop 14A in the lateral direction is derived from the coordinate values of both ends in the lateral direction of the tabletop 14A shown in the camera image. The total length of the tabletop 14A in the lateral direction is divided by the derived number of pixels to calculate the home position length-pixel conversion value LowTableMpp.

At the highest position of the tabletop 14A, the number of pixels corresponding to the entire length of the tabletop 14A in the lateral direction is derived from the coordinate values of both ends in the lateral direction of the tabletop 14A shown in the camera image. The total length of the tabletop 14A in the lateral direction is divided by the derived number of pixels to calculate the highest position length-pixel conversion value HighTableMpp.

From the measurement results, the linear function F₂ representing a relationship between the distance from the camera 27 to the tabletop 14A and the length-pixel conversion value is derived. A set of the slope a₂ and the intercept b₂ of the y-axis, which is a value axis, of the linear function F₂ is derived and stored as the front-rear lateral direction calibration data.

As a result, at any height of the tabletop 14A, the number of pixels from the estimated center position of the camera image to the scan start position in the camera image is converted into a distance in a unit such as millimeters.

[3]

In the lateral direction, the deviation amount UpTableZureX between the center position of the camera image at the home position of the tabletop 14A and the center position of the camera image at the highest position of the tabletop 14A is measured.

As a result, in the lateral direction, using the deviation amount UpTableZureX, the distance middle_line_mm from the center position of the camera image at any height of the tabletop 14A to the scan start position is converted into the distance high_table_middle_line_mm from the center position of the camera image at the highest position of the tabletop 14A to the scan start position.

In addition, in the front-rear direction, the deviation amount UpTableZureY between the center position of the camera image at the home position of the tabletop 14A and the center position of the camera image at the highest position of the tabletop 14A is measured.

As a result, in the front-rear direction, using the deviation amount UpTableZureY, the distance middle_line_st_mm from the center position of the camera image at any height of the tabletop 14A to the scan start position is converted into the distance high_table_middle_line_st_mm from the center position of the camera image at the highest position of the tabletop 14A to the scan start position.

[4]

At the highest position of the tabletop 14A, the distance ISOcenterDistanceX between the center of the camera image in the lateral direction and the ISO center is measured.

As a result, the moving distance move_shift_mm of the tabletop 14A in the lateral direction is calculated from the distance high_table_middle_line_mm from the center position of the camera image to the scan start position.

Similarly, at the highest position of the tabletop 14A, the distance ISOcenterDistanceY between the center of the camera image in the front-rear direction and the ISO center is measured.

As a result, the moving distance move_position_mm of the tabletop 14A in the front-rear direction is calculated from the distance high_table_middle_line_st_mm from the center position of the camera image to the scan start position.

[5]

For the minus side in the front-rear direction, a minus side measurement position separated from the center position of the camera image by a specified number of pixels is specified. The slope a₃ of the linear function F₃ on the minus side in the front-rear direction is specified by using the distance D_center from the camera 27 to the center position of the camera image, the distance D_minus from the camera 27 to the minus side measurement position, and the number of pixels from the center position of the camera image to the minus side measurement position.

As a result, even in a case in which the camera 27 is inclined to the minus side in the front-rear direction with respect to the installation surface PP, the moving distance of the tabletop 14A in the front-rear direction can be corrected based on the estimated value H_ₑₛₜᵢₘₐₜₑ of the distance from the camera 27 to the tabletop 14A at the center position of the camera image.

For the plus side in the front-rear direction, a plus side measurement position separated from the center position of the camera image by a specified number of pixels is specified. The slope a₄ of the linear function F₄ on the plus side in the front-rear direction is specified by using the distance D_center from the camera 27 to the center position of the camera image, the distance D_plus from the camera 27 to the plus side measurement position, and the number of pixels from the center position of the camera image to the plus side measurement position.

As a result, even in a case in which the camera 27 is inclined to the plus side in the front-rear direction with respect to the installation surface PP, the moving distance of the tabletop 14A in the front-rear direction can be corrected based on the estimated value H_ₑₛₜᵢₘₐₜₑ of the distance from the camera 27 to the tabletop 14A at the center position of the camera image.

### Regarding Program for Operating Computer

A program causing a computer to implement some or all of the processing functions of the examination control device 20 and the like according to the embodiment can be recorded in a computer-readable medium which is an optical disk, a magnetic disk, a semiconductor memory, or other tangible non-transitory information storage medium, and the program can be provided via the information storage medium.

Instead of the aspect of providing the program by storing the program in the tangible non-transitory computer-readable medium, it is also possible to provide the program as a program signal by using an electric communication line such as the Internet.

Further, some or all of the processing functions in the examination control device 20 or the like may be implemented by cloud computing, or may be provided as software as a service (SaaS).

### Example of Application to Medical Image Diagnostic Examination System

The measurement device 12 and the examination control device 20 shown in FIG. 1 may be electrically connected to each other via any network in a data communicable manner to constitute a medical image diagnostic examination system. The data communication between the measurement device 12 and the examination control device 20 may be wired communication or wireless communication.

The present disclosure is not limited to the above-described embodiment, and various modifications can be made without departing from the spirit of the technical idea of the present disclosure.

### EXPLANATION OF REFERENCES

10: MRI apparatus
12: measurement device
14: examination table
14A: tabletop
14D: side cover
16: console unit
17A: imaging room
20: examination control device
26: camera unit
27: camera
82: processor
84: memory
126: calibration data generation unit
130: camera image acquisition unit
132: camera image processing unit
134: distance information acquisition unit
136: distance information processing unit
138: actual measurement data acquisition unit
140: actual measurement data processing unit
a₁: slope
b₁: y-axis intercept
H_{high}: highest position camera-to-tabletop distance
H_{low}: home position camera-to-tabletop distance
M_{high}: highest position tabletop height
M_{low}: home position tabletop height
PP: installation surface
UpTableZureX: DX deviation amount
UpTableZureY: DY deviation amount
ZCN: correction value
ZCP: correction value

What is claimed is:

1. A calibration data generation device comprising:
one or more processors; and
one or more memories that store a program including one or more instructions executed by the one or more processors,
wherein the one or more processors execute the program stored in the one or more memories to
acquire, by using a sensor attached to a measurement room in which an examination table having a tabletop on which an examinee is placed is installed, first distance information representing a distance from the sensor to the tabletop at a first up-down position in an up-down direction orthogonal to an installation surface on which the examination table is installed,
acquire first plane information including information on a distance in a first plane orthogonal to the up-down direction at the first up-down position by using the sensor,
acquire second distance information representing a distance from the sensor to the tabletop at a second up-down position different from the first up-down position in the up-down direction by using the sensor,
acquire second plane information including information on a distance in a second plane orthogonal to the up-down direction at the second up-down position by using the sensor, and
generate calibration data applied to correction of a movement parameter of the tabletop in the up-down direction and a movement parameter of the tabletop in a plane orthogonal to the up-down direction at any position in the up-down direction, by using the first distance information, the first plane information, the second distance information, and the second plane information.

2. The calibration data generation device according to claim 1,
wherein the first up-down position corresponds to a home position where the tabletop is at its lowest, and
the second up-down position corresponds to a highest position where the tabletop is at its highest.

3. The calibration data generation device according to claim 1,
wherein the one or more processors acquire, as the calibration data, a combination of a slope and an intercept on a value axis of a linear function representing a relationship between a distance from the installation surface to the tabletop and a distance from the sensor to the tabletop.

4. The calibration data generation device according to claim 1,
wherein the one or more processors
acquire the first distance information and the second distance information measured using a distance measurement sensor that measures a distance to an object to be measured, and
acquire a captured image of the first plane as the first plane information and acquire a captured image of the second plane as the second plane information, from an imaging device that images the plane orthogonal to the up-down direction.

5. The calibration data generation device according to claim 4,
wherein the one or more processors acquire, as the calibration data, a distance per pixel in the captured image relative to a distance from the installation surface to the tabletop.

6. The calibration data generation device according to claim 4,
wherein the one or more processors acquire, as the calibration data, a distance between a center position of the captured image and a measurement center position of a measurement device that measures the examinee in a lateral direction orthogonal to a front-rear direction in which the tabletop moves forward or backward, for the second up-down position.

7. The calibration data generation device according to claim 4,
wherein the one or more processors acquire, as the calibration data, a distance between a center position of the captured image and a measurement start position specified for the examinee in a front-rear direction in which the tabletop moves forward or backward, for the second up-down position.

8. The calibration data generation device according to claim 4,
wherein the one or more processors acquire a captured image showing the entire tabletop as the captured image of the first plane.

9. The calibration data generation device according to claim 1,
wherein the one or more processors acquire, as the calibration data, a slope of a linear function representing a relationship between a position on the tabletop and a distance from the sensor to the tabletop.

10. A calibration data generation method comprising:
acquiring, by using a sensor attached to a measurement room in which an examination table having a placement surface on which an examinee is placed is installed, first distance information representing a distance from the sensor to a tabletop of the examination table on which the examinee is placed at a first up-down position in an up-down direction orthogonal to an installation surface on which the examination table is installed;
acquiring first plane information including information on a distance in a first plane orthogonal to the up-down direction at the first up-down position by using the sensor;
acquiring second distance information representing a distance from the sensor to the tabletop at a second up-down position different from the first up-down position in the up-down direction by using the sensor;
acquiring second plane information including information on a distance in a second plane orthogonal to the up-down direction at the second up-down position by using the sensor; and
generating calibration data applied to correction of a movement parameter of the tabletop in the up-down direction and a movement parameter of the tabletop in a plane orthogonal to the up-down direction at any position in the up-down direction, by using the first distance information, the first plane information, the second distance information, and the second plane information.

11. A non-transitory, computer-readable recording medium which records thereon a program for causing, when read by a computer, the computer to execute the calibration data generation method according to claim 10.

12. A medical image capturing apparatus comprising:
a measurement device that measures an examinee;
a measurement data processing device that generates a medical image of the examinee based on a measurement result of the examinee;
an examination table that is provided with a tabletop on which the examinee is placed;
a sensor that is attached to a measurement room in which the examination table is installed and that detects the examinee; and
a calibration data generation device that generates calibration data for correcting a movement parameter of the tabletop based on a position where the sensor is installed,
wherein the calibration data generation device includes
one or more processors, and
one or more memories that store a program including one or more instructions executed by the one or more processors, and
the one or more processors execute the program stored in the one or more memories to
acquire, by using the sensor attached to the measurement room in which the examination table on which the examinee is placed is installed, first distance information representing a distance from the sensor to the tabletop of the examination table on which the examinee is placed at a first up-down position in an up-down direction orthogonal to an installation surface on which the examination table is installed,
acquire first plane information including information on a distance in a first plane orthogonal to the up-down direction at the first up-down position by using the sensor,
acquire second distance information representing a distance from the sensor to the tabletop at a second up-down position different from the first up-down position in the up-down direction by using the sensor,
acquire second plane information including information on a distance in a second plane orthogonal to the up-down direction at the second up-down position by using the sensor, and
generate calibration data applied to correction of a movement parameter of the tabletop in the up-down direction and a movement parameter of the tabletop in a plane orthogonal to the up-down direction at any position in the up-down direction, by using the first distance information, the first plane information, the second distance information, and the second plane information.
